(19) United States
(12) Patent Application Publication
(10) Pub. No.: US 2020/0109117 A1
(43) Pub. Date: Apr. 9, 2020

(54) Modified Therapeutic Agent Analogs of Mefenamic Acid

(71) Applicant: ACCESS Association, Basel (CH)

(72) Inventor: Robert Derham, Basel (CH)

(21) Appl. No.: 16/590,980

(22) Filed: Oct. 2, 2019

Related U.S. Application Data
(60) Provisional application No. 62/739,993, filed on Oct. 2, 2018.

(51) Int. Cl. C07D 213/82; C07D 213/80

(52) U.S. Cl. CPC C07D 213/82; C07D 213/80

(57) ABSTRACT

The present disclosure provides certain compositions and methods useful in the treatment and/or prevention of a neurodevelopmental disorder, such as seizure disorders, autism or an autism spectrum disorder (ASD). More specifically, this includes compositions that contain at least one fenamate active agent, which are analogs to mefenamic acid and/or derivatives thereof, where such compositions exhibit increased solubility over MFA. The increased solubility provides for the potential for increased therapeutic action, when used in therapeutic treatment of Autism, Autism Spectrum Disorders (ASDs), seizures and neurodegenerative disorders over other known MFA analogs. In addition, these MFA analogs are effective in preventing an increase in permeability of the blood-brain barrier (BBB) without adversely altering brain metabolite levels, but at effective dosages restoring seizure effects and brain metabolites back to their normal levels.

### CROSS REFERENCE TO RELATED APPLICATION

The present application claims the benefit of U.S. patent application Ser. No. 62/739,993, filed Oct. 2, 2018, which is hereby incorporated by reference in its entirety.

### FIELD OF INVENTION

The present disclosure provides certain compositions and methods useful in the treatment and/or prevention of a neurodevelopmental disorder including seizure disorders, autism and/or an autism spectrum disorder (ASD). Compositions are provided that contain at least one fenamate active agent, such as mefenamic acid (MFA) analogs and/or derivatives thereof, where these compositions include one or more molecular structures that exhibit increased solubility in comparison with MFA. The increased solubility of these analogs compared with MFA provide the potential for increased therapeutic action, when used in the treatment of Autism, Autism Spectrum Disorders (ASDs), seizures and neurodegenerative disorders compared to other MFA analogs.

### BACKGROUND

It has been estimated that more than 40-60% of commercially available active pharmaceutical ingredients (APIs) are not sufficiently water soluble. For these drugs, poor aqueous solubility and poor dissolution correlates with low bioavailability after oral administration. Therefore, an improvement of physiochemical properties of poorly water-soluble drugs has become essential in drug development. Various techniques have been developed to improve drug solubility, such as nano-crystallization, salt formation, solid dispersion, complexation, nano-suspension and self-emulsifying drug delivery systems.

This disclosure covers the field of APIs. In particular, it relates to distinct structures based on anthranilic acid ester analogs, more specifically fenamates and most particularly mefenamic acid (MFA) analog or derivative compositions. Such compositions exhibit increased solubility over MFA, presented as structure (I), and therefore provide the potential for increased action, when used in therapeutic treatment of Autism, Autism Spectrum Disorders (ASDs), seizures and neurodegenerative disorders over previously presented MFA analogs.

More specifically these MFA analogs are effective in preventing an increase in permeability of the blood-brain barrier (BBB) without adversely altering brain metabolite levels, but at effective dosages restoring seizure effects and brain metabolites back to their normal levels.

Autism and ASDs include a broad range of developmental brain disorders that share a complex and heterogeneous etiology characterized by fundamental deficits in social reciprocity, impaired language and communication skills, as well as repetitive and stereotypic behavior. About 1% of the population are directly affected and of course many more family members must care for autistic children and adults

Every year, about 20,000 children develop symptoms of ASDs in the U.S. alone. The few drugs approved for treatment of autism merely address irritability associated with autism. Currently there are no approved pharmacological interventions to prevent regression to more severe forms of autism and ASDs. A child developing a life-long disability causes an approximately $10M burden on society in terms of lost income for patients and often one of the parents, as well as direct cost for treatment and assisted living. This situation leads to 20,000 children per year in need for life-long assistance in the U.S. alone and the lack of adequate treatment costs societies an estimated $200B per year. In addition, the social and emotional consequences of a life-long disability on the patient and his or her family are significant and immeasurable.

A seizure disorder is a medical condition (one of about 40 autistic individuals experience this) characterized by episodes of uncontrolled electrical activity in the brain, thus producing symptoms that include two or more seizures. There are approximately 40 named seizure disorders.

Experts propose that some of the developmental brain changes associated with autism also contribute to seizures. These differences in brain development appear to cause changes in the activity of brain nerve cells, or neurons. Neurons process and transmit information and send signals to the rest of the body. Certain disturbances in their activity can cause seizures.

Epilepsy is a brain disorder marked by recurring seizures, or convulsions. It affects a fifth to a third of people who have autism, compared to an estimated 1 to 2 percent of the general population (Spence, S. Pediatric Research Vol. 65, pages 599-606 (2009)). The autism-epilepsy overlap appears to be most common among people who also have intellectual disability. (Amiet, C. Biol Psychiatry. 2008 Oct. 1; 64(7):577-82)

Identifying and effectively treating epilepsy is critically important, given the potential for brain damage and death from uncontrolled seizures. While the association between epilepsy and autism is well known, diagnosis can be challenging because seizures are not always outwardly evident, and many people with autism have difficulty recognizing and communicating their symptoms.

Seizures can begin at any age, though research has identified two peaks in onset among children with autism—in the preschool years and again in adolescence. Like autism, epilepsy exists along a spectrum. Severity varies widely. In addition, experts now distinguish seizures by the location of onset in the brain. This is important because it affects the choice of seizure medication, the potential benefit of epilepsy surgery, future outcomes and possible causes.

Mefenamic acid, N-(2,3-Dimethylphenyl)anthranilic acid, is a member of the anthranilic acid derivatives (or fenamate) class of NSAID drugs, and is used to treat mild to moderate pain, including menstrual pain. It is also sometimes used to prevent migraines associated with menstruation. Like other members of the fenamate class of NSAID drugs, it inhibits both isoforms of COX and prevents formation of prostaglandins. MFA is not widely used in the United States, due to its side effects and high cost compared to other NSAID drugs. The chemical structure of MFA is shown below as Structure (I);

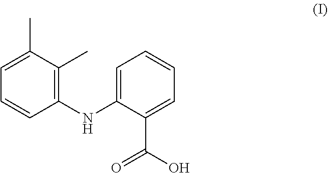 [SMILES: Cc1cccc(Nc2ccccc2C(=O)O)c1C]

MFA is an example of a fenamate that is currently being used safely and effectively in human subjects, including children, and that could be repurposed for use in the compositions and methods of the present invention, for example for the treatment of seizure disorders, autism or ASDs. In some countries of the European Union (EU), mefenamic acid is approved for use in children from 6 months of age for the treatment of pain and fever, including chronic use in juvenile arthritis. Mefenamic acid is also available in specific pediatric formulations (oral suspension) in the EU and suitable dosing regimens in infants and children, as well as adults, are established.

Methods of producing Mefenamic acid (N-(2,3-Dimethylphenyl)anthranilic acid) are well known. Any suitable method of making MFA can be used, some examples of which are provided herein and also incorporated by reference in their entirety.

U.S. Pat. No. 3,138,636 relates to novel anthranilic acids having useful pharmacodynamic properties and to methods for producing same. More particularly, the present disclosure relates to anthranilic acids and salts thereof, these anthranilic acids having in their free acid form the formula shown below;

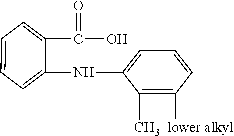

The molecular structures wherein the lower alkyl is methyl or ethyl possess exceptionally high activity and are the preferred molecular structures of the present disclosure. U.S. Pat. No. 3,313,848 provides details regarding halogenated derivatives.

U.S. Pat. No. 4,135,050 provides an improved process for preparing anthranilic acid esters of formula;

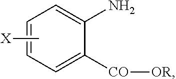

wherein X represents a hydrogen or halogen atom, preferably a chlorine or bromine atom, a hydroxy group, an alkyl radical having from 1 to 4 carbon atoms, an alkoxy radical having from 1 to 4 carbon atoms or a nitro group and R represents a linear or branched alkyl radical having from 2 to 5 carbon atoms, by transesterification of an anthranilic acid alkyl ester in the presence of a basic catalyst.

Mechanisms of action for fenamate analogs, and more specifically MFA, are known. Any suitable mechanism of action can be targeted in which the treatment selected is used in therapeutic treatment of neurodevelopmental disorders such as Autism and Autism Spectrum Disorders (ASDs), seizures and neurodegenerative disorders, some examples of which are provided herein and also incorporated by reference in their entirety.

US 2016/0206581 A1provides certain compositions and methods that may be useful in the treatment and/or prevention of a neurodevelopmental disorder, such as autism or an autism spectrum disorder (ASD). Such methods aim at improving one or more indicators or symptoms of autism or an ASD in a human subject, where the method comprises administering to a subject exhibiting one or more indicators or symptoms of autism or an ASD, or a subject at risk of developing autism or an ASD, an effective amount of a fenamate, It can be also be fenamate or an analog or derivative thereof. In some such embodiments, compositions are provided that contain at least one fenamate active agent, such as mefenamic acid, or an analog or derivative thereof. In some embodiments, such compositions may also comprise an additional active agent, such as gabapentin, or an analog or derivative thereof, where the active agents target ion channels and/or modulate their activity.

An understanding of the genetic risk factors underlying seizure disorders, autism and ASDs can be useful for defining drug targets and developing therapeutic molecular structures and treatment methods which target the mechanisms underlying seizure disorders, such as epilepsy, autism and ASDs. The present disclosure provides MFA analogues that instead of only treating symptoms provide, therapeutic approaches for seizure disorders, autism and ASDs. These analogues can be developed to prevent the onset of disease, delay the progression of disease, and/or cure the disease.

### SUMMARY

Some of the main aspects of the present invention are summarized below. Additional aspects of the present invention are described in the Detailed Description, Examples, Drawings and the Claims sections of this disclosure. The description in each of the sections of this patent application is intended to be read in conjunction with the other sections. Furthermore, the various embodiments described in each of the sections of this disclosure can combined in various different ways, and all such combinations are intended to fall within the scope of the present disclosure.

In one aspect, the present disclosure provides a method of treating seizure disorders, autism or an ASD in a subject, the method comprising administering to the subject an effective amount of a fenamate such as a mefenamic acid (MFA) analog or derivative thereof, either alone or in combination with one or more additional active agents. For example, in one embodiment, the present disclosure provides a method of treating a seizure disorder, autism or an ASD in a subject, the method comprising administering to the subject an effective amount of an analog or derivative of the fenamate mefenamic acid (MFA), either alone or in combination with one or more additional active agents and or additional MFA analogs or derivatives provided herein, wherein the compositions selected have increased solubility over MFA alone and are effective in preventing an increase in permeability of the blood-brain barrier (BBB) without adversely altering brain metabolite levels, but at effective dosages restore seizure effects and brain metabolites to normal levels.

One embodiment provides a method of treating seizure disorders, autism or an ASD in a subject, the method comprising administering to the subject an effective amount of an MFA analog or derivative composition alone or in combination with an effective amount of an at least one additional MFA analog or derivative composition, wherein each additional MFA analog or derivative composition is different than each of the other MFA analog or derivative compositions selected.

In one aspect, this disclosure provides a method of improving one or more indicators or symptoms of autism or an ASD in a subject, the method comprising administering to a subject exhibiting one or more indicators or symptoms of autism or an ASD, an effective amount of a fenamate analog or derivative thereof, either alone or in combination with one or more additional active agents or additional fenamate analogs or derivatives thereof, wherein the indicator is selected from the group consisting of abnormal behavior, abnormal eye tracking response, abnormal skin conductance response, abnormal electroencephalography (EEG) response, and/or abnormal magnetoencephalography (MEG) response. In one embodiment, “improving” comprises an increase of at least 1% in a measurement of the one or more indicators or symptoms. For example, another embodiment provides a method of improving one or more indicators or symptoms of autism or an ASD in a subject, the method comprising administering to a subject exhibiting one or more indicators or symptoms of autism or an ASD an effective amount of the MFA analog or derivative compositions, either alone or in combination with one or more additional active agents or MFA analogs or derivative compositions.

In yet a further embodiment an additional active agent may be selected from the group consisting of: ion channel modulators or an analog or derivative thereof, vitamins, such as methylated forms of Vitamin B₃ (Nicotinamide, see Structure (II)), B₆, B₉, B₁₂ or an analog or derivative thereof, and where the analog or derivative of B₃ includes N,N-Dimethylnicotinamide (provided as Structure (III), N-Methylnicotinamide (provided as Structure (IV), N-Methylnicotinic acid (provided as Structure (V), or N-(1-methylethyl) carbamic acid] (provided as Structure (VI).

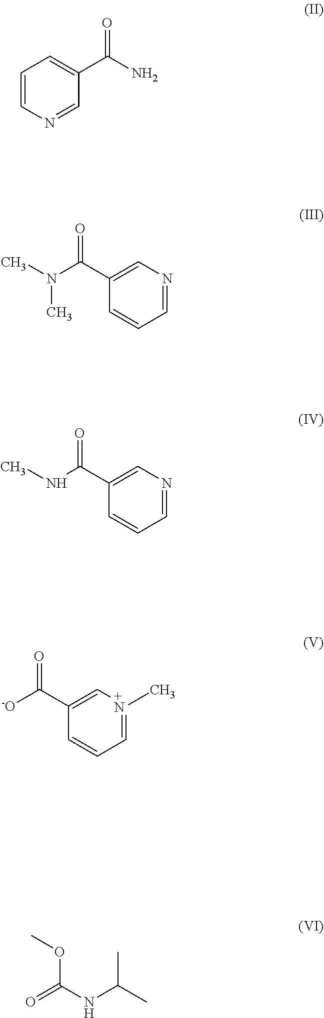

In another embodiment, such an additional active agent may be a curcuminoid or curcuminoid metabolite, where the preferred metabolite is tetrahydrocurcumin (THC) as provided in Structure (VIII).

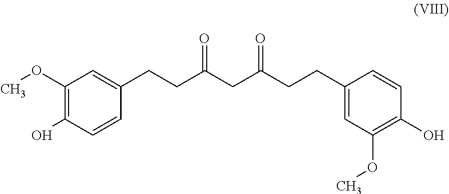 [SMILES: COc1cc(CCC(=O)CC(=O)CCc2ccc(O)c(OC)c2)ccc1O]

A curcuminoid is a linear diarylheptanoid, with molecules such as curcumin or derivatives of curcumin with different chemical groups that have been formed to increase the solubility of curcumins and make them suitable for drug formulation. These molecular structures are natural phenols and produce a pronounced yellow color.

Many curcumin characters are unsuitable for use as drugs by themselves. These molecular structures exhibit poor solubility in water at acidic and physiological pH, and also hydrolyze rapidly in alkaline solutions. Therefore, curcumin derivatives are synthesized to increase their solubility and hence bioavailability Tetrahydrocurcumin, one of the main metabolites of curcumin, is the most potent antioxidant among the naturally occurring curcuminoids. The curcuminoids are capable of inhibiting damage to super coiled plasmid DNA by hydroxyl radicals. It was concluded that the derivatives of curcumin are good in trapping the 2,2-diphenyl-1-picrylhydrazyl (DPPH) radical as efficiently as curcumin which is a well- known antioxidant.

In an additional embodiments, the molecular structures utilizing various B-vitamins are used in combination with molecular structures complexed with curcuminoid or curcuminoid metabolites, as the mechanisms of action differ and combined use can provide a concomitant effect.

In an additional embodiment, a method of improving one or more indicators or symptoms of autism or an ASD in a subject while normalizing the effects of seizure disorder(s) exists. In this case, the method comprises administering to a subject exhibiting one or more indicators or symptoms of autism or an ASD or seizure disorder, an effective amount of an MFA analog or derivative composition alone or in combination with an effective amount of another MFA analog or derivative composition and/or an active ingredient. In a further embodiment, the active ingredient can be used in conjunction with the MFA analog or derivative or can be used to create the MFA analog or derivative.

In yet other embodiments, the fenamate used in the various methods and compositions described herein is selected from the group consisting of fenamic acid, mefenamic acid (MFA), tolfenamic acid (TFA), flufenamic acid (FFA), meclofenamic acid (CFA), and analogs and derivatives thereof. In the preferred embodiments, the fenamate is MFA.

In yet other embodiments, the MFA analog, is administered to the subject at a dose of about 1 mg to about 500 mg per day. In some cases the MFA analog, is administered to the subject at a dose of about 25 mg to about 75 mg per day. Here it is also possible for the MFA analog to be administered to the subject at a dose of between at least 0.1 mg and up to at least 200 mg per day. In other cases the MFA analog, is administered to the subject at a dose in the range of 1 to 1000 mg per day. In some cases, each of the dosages described above is mg/kg/day. Additional dosages that may be used are provided in the Detailed Description section.

The present disclosure also provides for administering to a subject one or more active agents selected from the group consisting of potassium channel modulators, chloride channel modulators (including CaCCs), and calcium channel modulators. In some embodiments, this disclosure comprises administering to a subject both a fenamate, or an analog or derivative thereof, and one or more additional active agents selected from the group consisting of potassium channel modulators, chloride channel modulators (including CaCCs), and calcium channel modulators. In further embodiments the disclosure comprises administering to a subject both a fenamate, or an analog or derivative thereof, and an additional active agent that is a potassium or chloride or other salt ions acting as channel modulators. In some other embodiments, the present disclosure comprises administering to a subject both a fenamate, or an analog or derivative thereof, and two or more additional active agents selected from the group consisting of calcium channel modulators, chloride channel modulators, and potassium channel modulators. Where calcium channel modulators are used, the calcium channel modulator may be selected from the group consisting of gabapentin, pregabalin, or atagabalin, or an analog or derivative thereof.

This disclosure also provides for a method of treating a neurodevelopmental disease or disorder, such as autism or an ASD, in a subject, the method comprising administering to the subject an effective amount of one or more chloride channel modulators. In addition, a method of treating a neurodevelopmental disease or disorder, such as autism or an ASD, in a subject is disclosed. One method comprises administering to the subject an effective amount of a CaCC modulator. The disclosure also includes a method of treating a neurodevelopmental disease or disorder, such as autism or an ASD, in a subject, the method comprising administering to the subject an effective amount of a calcium channel modulator or both an effective amount of both a chloride channel modulator and a potassium channel modulator and/or a calcium channel modulator.

Where the CaCC modulators are used, the CaCC modulator is a modulator of an anoctamin CaCC. The CaCC modulator can be a modulator of an ANO1, ANO2, ANO3, ANO4, ANO5, ANO6, ANO7, ANO8, ANO9, or ANO10 anoctamin CaCC. The CaCC modulator is a fenamate, or an analog or derivative thereof. Here, the CaCC modulator is selected from the group consisting of fenamic acid, MFA, TFA, FFA, NFA and CFA, and analogs and derivatives thereof. In some embodiments, the CaCC modulator is MFA or an analog or derivative thereof, and is administered to the subject at a dose of about 10 mg/kg/day to about 200 mg/kg/day, or one or the other dosages or dosage ranges described above and/or in the Detailed Description section of this patent application.

It is also true that the potassium channel modulator is a fenamate analog or derivative thereof. In some embodiments, the potassium channel modulator is selected from the group consisting of fenamic acid, MFA, TFA, FFA, NFA and CFA, and analogs and derivatives thereof. It also noted that the potassium channel modulator is MFA or an analog or derivative thereof.

This disclosure also provides methods that allow for administering to the subject a voltage-activated Ca²+channel modulator—in addition to a chloride channel and/or potassium channel modulator as described above (such as a fenamate). In yet another embodiment, the voltage-operated calcium channel (VOCC) modulator is gabapentin, pregabalin, or atagabalin, or an analog or derivative thereof. Thus in one embodiment the present invention provides, a method of treating a neurodevelopmental disease or disorder, such as autism or an ASD in a subject, the method comprising administering to the subject an effective amount of gabapentin, pregabalin, or atagabalin, or an analog or derivative thereof.

In some embodiments, the subjects treated using the compositions or methods described herein, are human. In some embodiments, the subject may be a human child of any age, including a newborn. In another embodiment, the subject may be a human child of at least 1 month, 2 months, 3 months, 4 months, 5 months, 6 months, 9 months, or 12 months in age. In another embodiment, the subject is a human child of less than about 36 months (3 years) in age. In another embodiment, the subject is a human child of less than about 24 months (2 years) in age. Additional age ranges of the subjects of the invention are provided in the Detailed Description section of the patent application.

In some embodiments, the subjects treated according to the methods described herein, or treated using the compositions described herein, exhibit one or more clinical indicators or symptoms of epilepsy, autism or an ASD. In other embodiments, the subjects do not exhibit one or more clinical indicators or symptoms of epilepsy, autism or an ASD and the treatment methods are prophylactic treatment methods. In some embodiments, the subjects have been identified as being at risk of developing epilepsy, autism or an ASD, for example as the result of a family history of epilepsy, autism or an ASD or as the result of genetic testing, for example for a mutation believed to be associated with epilepsy, autism or an ASD. In some embodiments, the subject has a family history of epilepsy, autism. In some embodiments, the subject has one or more genetic risk factors associated with epilepsy, autism and/or ASDs. In some embodiments, the subject has a genetic mutation which can include a mutation in a receptor protein tyrosine phosphatase (PTPR). The methods of the disclosure also comprise conducting genetic testing on a subject, or obtaining genetic testing results of a subject, in order to determine if a subject is at risk of developing seizure disorders, autism or an ASD. Genetic testing may be performed, or such genetic testing results may be obtained, prior to initiating treatment of the subject using any of the methods and/or compositions provided herein, and/or in order to determine whether the subject is a candidate for treatment using any of the methods and/or compositions provided herein.

This disclosure also provides the use of various compositions, including pharmaceutical compositions. In some embodiments such compositions can be used in conjunction with the methods of treatment provided herein. For example, in one embodiment, pharmaceutical compositions comprising any one or more of the active agents described herein, either alone or in combination, for example for use in treating seizure disorders, autism or an ASD are provided. Further embodiments provide for a fenamate analog or derivative thereof, for example for use in treating seizure disorders, autism or an ASD. This disclosure provides compositions comprising nicotinamide, N,N-Dimethylnicotinamide, N-Methylnicotinamide, N-Methylnicototinic acid, N-(1-methylethyl) carbamic acid, tetrahydrocurcumin (THC), vitamin B₆, B₉, B₁₂ or an analog or derivative thereof, for example for use in treating seizure disorders, autism or an ASD.

Analogs of fenamic acid of the embodiments herein are provided by the following structures:

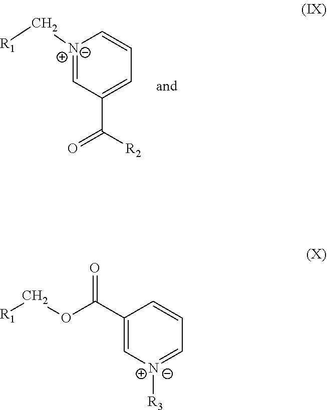

where R₁ is selected from the group consisting of anthranilic acid esters, to include fenamic acid, MFA, tolfenamic acid (TFA), flufenamic acid (FFA), meclofenamic acid (CFA), and analogs and derivatives thereof;

R₂ is selected from the group consisting of an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine, 2,2′-oxybis ethanamine, alanine, and lysine or derivatives thereof;

R₃ is selected from the group consisting of a lower alkyl, an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, N-(1-methylethyl) carbamic acid, alanine, and lysine or derivatives thereof;

Additional embodiments of Structure (IX) include:

Structure (IX) where R₁=MFA and R₂=—NH2, which yields an MFA-Nicotinamide molecular structure provided as MFA-NIC which is shown as Structure (XI) below;.

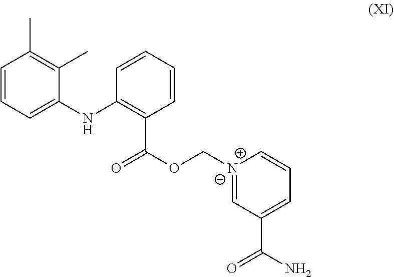

In addition Structure (IX) where R₁=MFA and R₂=—N(CH₃)₂, yields a MFA- N,N-Dimethylnicotinamide N-Methylnicotinamide molecular structure provided as MFA-2MeNIC and now shown below as Structure (XII).

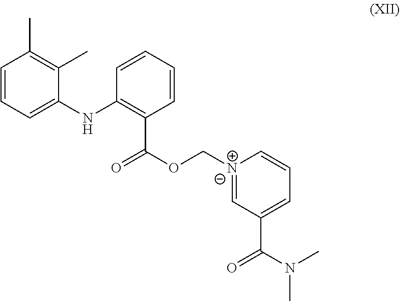

Another analogue for structure (IX) where R₁=MFA and R₂=—NHCH₃, yields a MFA- N-Methylnicotinamide molecular structure provided as MFA-MeNIC and is shown as Structure (XIII) below;

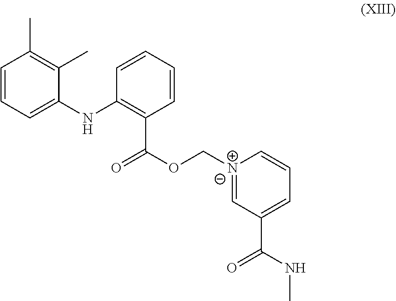

Additional analogs of Structure (X) include:

Structure (X) where R₁=MFA and R₃=—CH₃, yielding a MFA-N-methylnicotinic acid molecular structure provided as MFA-MeNICA and shown below as Structure (XIV).

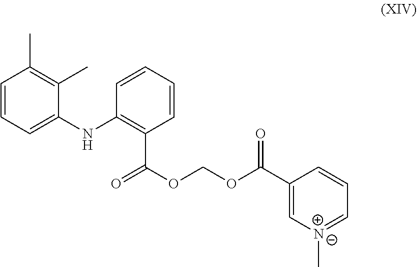

In addition, another analog for structure (X) is shown where R₁=MFA and R₃=N-(1-methylethyl) carbamic acid, which yields a MFA-Pyridinium, 3-carboxy-1-[N-(1-methylethyl) carbamic acid] provided as MFA-PCA and shown as Structure (XV).

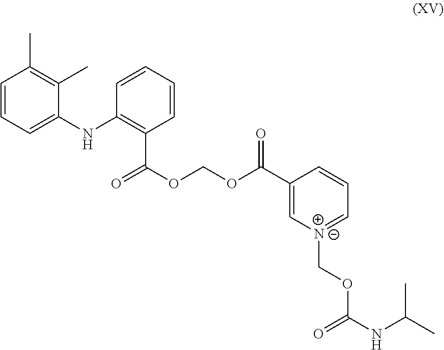

Additional embodiments provide compositions comprising both a fenamate and analogs or derivatives thereof, and a calcium channel modulator, such as gabapentin, pregabalin, or atagabalin, or an analog or derivative thereof, for example for use in treating autism or an ASD. A non-limiting example of such a composition is one comprising MFA analogs or derivative thereof and gabapentin or an analog or derivative thereof.

In other embodiments, the fenamate analog or derivative thereof is selected from the group consisting of MFA-NIC, MFA-MeNIC, MFA-2MeNIC, MFA-MeNICA and MFA-PCA.

In addition to all of the classes of molecules and specific molecules described herein as potential active agents, derivatives and analogs of such molecules/agents can be used in the compositions and methods of the present invention. Derivatives of the molecules/agents described herein that can be used in accordance with the compositions and methods of the present invention include, but are not limited to, prodrug derivatives.

Also, prodrug derivatives may comprise polyethylene glycol molecules that have been covalently attached to the drug molecule (i.e. pegylated derivatives). In further embodiments such prodrugs may comprise, for example, two (or more) active agent molecules connected together, either directly or by a linker (or linkers) that can be degraded inside the body, such as a disulfide linker, palmityl linker, stearyl linker, glycol linker, polyethylene glycol linker, or ester linker. These prodrugs may comprise two (or more) of the same active agent molecule, such as two MFA molecules connected together or analogs thereof, either directly or by a linker such as a disulfide linker, palmityl linker, stearyl linker, glycol linker, polyethylene glycol linker, or ester linker. In some embodiments such prodrugs may comprise two (or more) different active agent molecules, such as an MFA analog and a nicotinamide molecule, connected together, either directly or by a linker such as a disulfide linker, palmityl linker, stearyl linker, glycol linker, polyethylene glycol linker, or ester linker. In some embodiments such prodrugs may comprise an active agent molecule and any another suitable molecule, moiety, or chemical group (whether an active agent or not) that can be cleaved or removed from the prodrug to release the active agent molecule.

In many embodiments, the present disclosure provides certain MFA analog prodrug molecules as well as pharmaceutical compositions, comprising the MFA analog prodrugs. In all embodiments, the MFA prodrugs or analogs or derivatives thereof, may be used to treat seizure disorders, autism or an ASD as described herein. In some embodiments, the MFA prodrugs or analogs or derivatives thereof, maybe used in any method or situation in which MFA can be used—i.e. not limited to treatment of seizure disorders, autism and ASDs.

### BRIEF DESCRIPTION OF THE DRAWINGS

FIG. 1. Chromatogram displaying stability of Structure (XI), MFA-NIC, on Day 7 at room temperature.

FIG. 2. Chromatogram displaying stability of Structure (XII), MFA-MeNIC, on Day 7 at room temperature.

FIG. 3. Chromatogram displaying stability of Structure (XIII), MFA-2MeNIC, on Day 7 at room temperature.

FIG. 4. BBB Permeability measurements in the hippocampus brain region of saline controls, pilocarpine-induced seizure rats, and MFA- or pro-MFA-treated rats with pilocarpine-induced seizures, both at various doses (10, 50 or 200 mg/kg via gavage). There was a significant increase in BBB permeability in the MFA 50 mg/kg group compared to saline controls. ***p<0.001.

FIG. 5. BBB Permeability measurements in the thalamus brain region of saline controls, pilocarpine-induced seizure rats, and MFA- or pro-MFA-treated rats with pilocarpine-induced seizures, both at various doses (10, 50 or 200 mg/kg via gavage). There was a significant increase in BBB permeability in the MFA 50 mg/kg group compared to saline controls. *p<0.05, **p<0.01.

FIG. 6. BBB Permeability measurements in the cerebral cortex brain region of saline controls, pilocarpine-induced seizure rats, and MFA- or pro-MFA-treated rats with pilocarpine-induced seizures, both at various doses (10, 50 or 200 mg/kg via gavage). There was a significant increase in BBB permeability in the MFA 50 mg/kg group compared to saline controls. *p<0.05, **p<0.01, ***p<0.001.

FIG. 7. NAA/Cho metabolite ratio measurements in the central brain region (primarily hippocampus) of controls (non-treated, non-seizure), pilocarpine-induced seizure rats, and MFA- or pro-MFA-treated rats with pilocarpine-induced seizures, both at various doses (10, 50 or 200 mg/kg via gavage). There was a significant decrease in NAA/Cho ratios in the MFA 200 mg/kg and pro-MFA 10 mg/kg groups compared to saline

FIG. 8. Glu/Cho metabolite ratio measurements in the central brain region (primarily hippocampus) of controls (non-treated, non-seizure), pilocarpine-induced seizure rats, and MFA- or pro-MFA-treated rats with pilocarpine-induced seizures, both at various doses (10, 50 or 200 mg/kg via gavage).

FIG. 9. Cr/Cho metabolite ratio measurements in the central brain region (primarily hippocampus) of controls (non-treated, non-seizure), pilocarpine-induced seizure rats, and MFA- or pro-MFA-treated rats with pilocarpine-induced seizures, both at various doses (10, 50 or 200 mg/kg via gavage). There was a significant decrease in Cr/Cho ratios in the MFA 10 mg/kg group compared to saline controls. *p<0.05.

FIG. 10. Tau/Cho metabolite ratio measurements in the central brain region (primarily hippocampus) of controls (non-treated, non-seizure), pilocarpine-induced seizure rats, and MFA- or pro-MFA-treated rats with pilocarpine-induced seizures, both at various doses (10, 50 or 200 mg/kg via gavage).

FIG. 11. Myo-Ins/Cho metabolite ratio measurements in the central brain region (primarily hippocampus) of controls (non-treated, non-seizure), pilocarpine-induced seizure rats, and MFA- or pro-MFA-treated rats with pilocarpine-induced seizures, both at various doses (10, 50 or 200 mg/kg via gavage). There was a significant decrease in Myo-Ins/Cho ratios in the pro-MFA 10 mg/kg group compared to saline controls. *p<0.05.

FIG. 12. Results of an electroencephalogram (EEG) for the control, seizure, and MFA analog treatment rat of the accepted seizure model.

### DETAILED DESCRIPTION

Many of the embodiments of the present disclosure are described in the above Summary section of this application, as well as in the Examples, Figures, and Claims. This Detailed Description section provides additional description relating to the compositions and methods of the present invention, and is intended to be read in conjunction with all other sections of the present patent application, including the Summary of the Invention, Examples, Figures, and Claims sections of the present disclosure but not limiting the breadth and scope of the disclosure.

I. Abbreviations & Definitions

The abbreviation “APAP” refers to acetaminophen/paracetamol.

The abbreviation “API” refers to active pharmaceutical ingredient.

The abbreviation “ASA” refers to acetylsalicylic acid.

The abbreviation “ASD” refers to Autism Spectrum Disorder.

The abbreviation “BBB” refers to blood-brain barrier.

The abbreviation “CaCC” refers to Ca^{2±}-activated Cl⁻ channel.

The abbreviation “CAE” refers to childhood absence epilepsy.

The abbreviation “CE” refers to Contrast-enhanced.

The abbreviation “CE-MRI” refers to contrast enhanced magnetic resonance imaging.

The abbreviation “CFA” refers to meclofenamic acid.

The abbreviation “Cho” refers to choline.

The abbreviation “Cr” refers to creatine.

The abbreviation “EEG” refers to electroencephalography.

The abbreviation “FFA” refers to flufenamic acid.

The abbreviation “GABA” refers to gamma-aminobutyric acid.

The abbreviation “Gd-DTPA” refers to gadolinium diethylene triamine penta acetic acid.

The abbreviation “GI” refers to gastrointestinal.

The abbreviation “Glu” refers to glutamate.

The abbreviation “HFA” refers to high-functioning autism.

The abbreviation “MEG” refers magnetoencephalography.

The abbreviation “MFA” refers to mefenamic acid.

The abbreviation “MR” refers to magnetic resonance.

The abbreviation “MRI” refers to magnetic resonance imaging.

The abbreviation “MRS” refers to magnetic resonance spectroscopy.

The abbreviation “Myo-Ins” refers to myo-inositol.

The abbreviation “NAA” refers to N-acetyl aspartate.

The abbreviation “NFA” refers to niflumic acid.

The abbreviation “NSAID” refers to non-steroidal anti-inflammatory drug.

The abbreviation “SDA” refers to strict definition autism.

The abbreviation “SE” refers to status epilepticus.

The abbreviation “Tau” refers to taurine.

The abbreviation “TD” refers to typical development

The abbreviation “TFA” refers to tolfenamic acid.

The abbreviation “VOCC” refers to voltage-operated Ca²⁺ channels.

The abbreviation “VOPC” refers to voltage-operated potassium channels.

As used herein, the terms “about” and “approximately,” when used in relation to numerical values, mean within +or −20% of the stated value.

As used herein, the terms “treat,” “treating,” and “treatment” encompass a variety of activities aimed at desirable changes in clinical outcomes. For example, the term “treat”, as used herein, encompasses any activity aimed at achieving, or that does achieve, a detectable improvement in one or more clinical indicators or symptoms of a neurodevelopmental disease or disorder, such as autism or an ASD. For example, such terms encompass alleviating, abating, ameliorating, relieving, reducing, inhibiting, preventing, or slowing at least one clinical indicator or symptom, preventing additional clinical indicators or symptoms, reducing or slowing the progression of one or more clinical indicators or symptoms, causing regression of one or more clinical indicators or symptoms, relieving a condition caused by the disease or disorder, and the like. As used herein the terms “treat,” “treating,” and “treatment” encompass both preventive/prophylactic treatments and therapeutic treatments. In the case of prophylactic treatments, the methods and compositions provided herein can be used preventatively in subjects that do not yet exhibit any clear or detectable clinical indicators or symptoms of the disease or disorder but that are believed to be at risk of developing the disease or disorder, such as seizure disorders, autism or an ASD, for example as a result of family history or as a result of genetic testing. In the case of therapeutic treatments, the methods and compositions provided herein can be used in subjects that already exhibit one or more clinical indicators or symptoms of the disease or disorder, such as seizure disorders, autism or an ASD. In the case of autism and ASDs, various clinical indicators and symptoms are known to medical practitioners and those of skill in the art. Such symptoms include, but are not limited to, changes in eye tracking, skin conductance and/or EEG measurements in response to visual stimuli, difficulties engaging in and responding to social interaction, verbal and nonverbal communication problems, repetitive behaviors, intellectual disability, difficulties in motor coordination, attention issues, sleep disturbances, and physical health issues such as gastrointestinal disturbances.

The term “autism” is used herein in accordance with its usual usage in the art and includes, but is not limited to, SDA, HFA, and other ASDs. ASD and autism are both terms that encompass a group of complex disorders of brain development. These disorders include, but are not limited to, autistic disorder, Rett syndrome, childhood disintegrative disorder, pervasive developmental disorder not otherwise specified (PDD-NOS), and Asperger's syndrome.

The terms “epilepsy” and “seizure disorder” are used herein in accordance with usual usage in the art and includes, but is not limited to, SE, CAE, and other seizure disorders. Epilepsy and seizure disorder are both terms that encompass a group of excessive and abnormal brain cell activity.

The term “subject” as used herein encompasses mammals, including, but not limited to, humans, non-human primates, rodents (such as rats, mice and guinea pigs), and the like. In some embodiments of the invention, the subject is a human.

The terms “effective amount” or “therapeutically effective amount,” as used herein, refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as a dose escalation study.

The term “pharmaceutical composition” as used herein refers to a composition comprising at least one active agent as described herein (such as, for example, a fenamate analog or derivative, a calcium-activated chloride channel modulator, a potassium channel modulator, a voltage-activated calcium channel modulator, vitamin, curcuminoid or curcuminoid metabolite, etc.), or a combination of two or more active agents, and one or more other components suitable for use in pharmaceutical delivery such as carriers, stabilizers, diluents, dispersing agents, suspending agents, thickening agents, excipients, and the like.

The term “active agent” as used herein refers to a molecule that is intended to be used in the compositions and methods described herein and that is intended to be biologically active, for example for the purpose of treating seizure disorders, autism or an ASD. The term “active agent” is intended to include molecules that either are, or can be converted to a form that is, biologically active. For example, the term “active agent” includes pro-drugs and/or molecules that are inactive or lack the intended biological activity but that can be converted to a form that is active or has the intended biological activity.

Additional definitions and abbreviations are provided elsewhere in this patent specification or are well known in the art.

The MFA analogs, herein including at least MFA-NIC, MFA-MeNIC, MFA-2MeNIC, MFA-MeNICA and MFA-PCA, were subjected to stability and solubility studies in order to determine the ability of the modified drug to undergo pre-clinical trials.

Solubility

All five therapeutic MFA analogs were kept at room temperature (˜25° C.) and at 4° C. for one day and for 7 days. Therapeutic agents stable after 1 day were subjected to a 7 day stability study. Stability of the modified MFA analogs was checked by HPLC once visual confirmation of stability after 7 days.

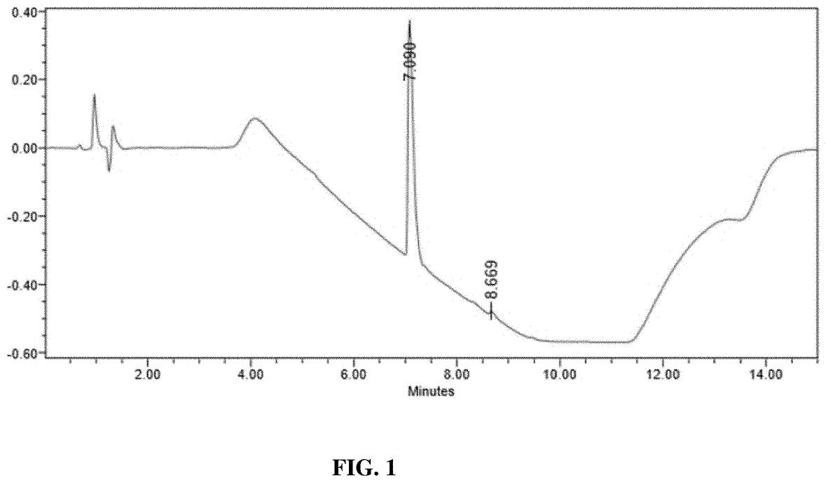

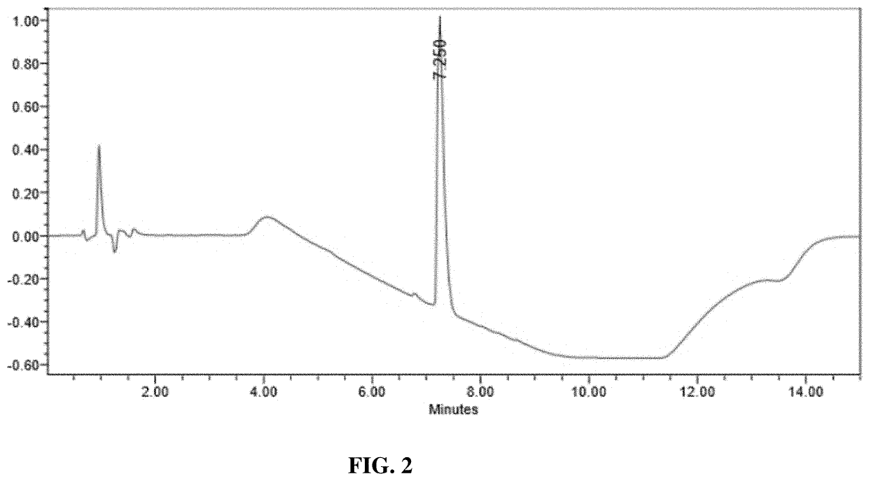

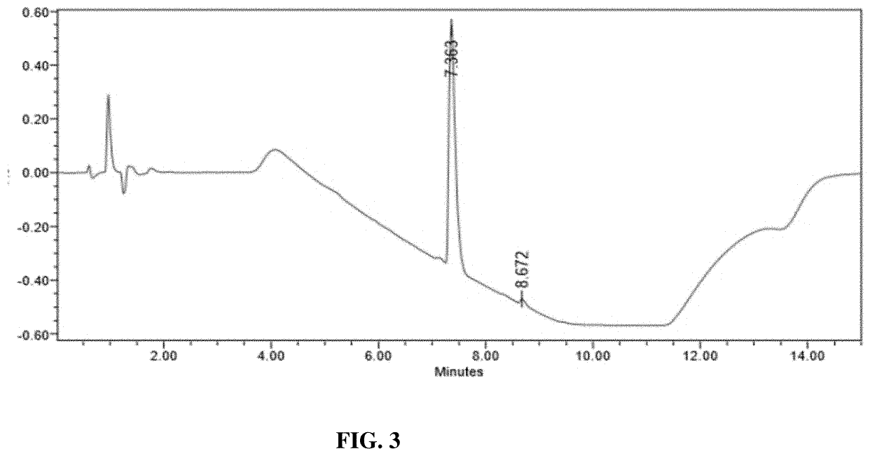

MFA-NIC, MFA-MeNIC and MFA-2MeNIC were found to be stable in acetonitrile and as well as in distilled water (DW) up to 7 days when kept at 4° C. and at room temperature (˜25° C.). FIG. 1 provides the HPLC chromatogram of MFA-NIC showing stability of the modified drug kept at room temperature on day seven. FIG. 2 provides the HPLC chromatogram of MFA-MeNIC showing stability of the modified drug kept at room temperature on day seven. FIG. 3 provides the HPLC chromatogram of MFA-2MeNIC showing stability of the modified MFA analog kept at room temperature on day seven. MFA-MeNICA was found to be stable in acetonitrile but was found to be unstable in distilled water after 1 day and hence not subjected to the 7 days study. MFA-PCA was not found to be stable in acetonitrile or in distilled water after 1 day, hence not subjected to the 7 days study. The results of the stability study have been summarized in Table 1.

As stability of a new analog could be found under alternative conditions or with additional modifications, the MFA-MeNICA and MFA-PCA molecular structures are still determined to be viable therapeutic agents and further testing is beign conducted.

TABLE 1
Modified Drugs OF MFA—7 day Solubility Data

| | 1 Day | | | | 7 Day | | | |
|---|---|---|---|---|---|---|---|---|
| | Molecular structure | | | | | | | |
| | ACN | ACN | DW | DW | ACN | ACN | DW | DW |
| | 4° C. | RT | 4° C. | RT | 4° C. | RT | 4° C. | RT |
| MFA- | Stable | Stable | Stable | Stable | Stable | Stable | Stable | Stable |
| NIC | | | | | | | | |
| MFA- | Stable | Stable | Stable | Stable | Stable | Stable | Stable | Stable |
| MeNIC | | | | | | | | |
| MFA- | Stable | Stable | Stable | Stable | Stable | Stable | Stable | Stable |
| 2MeNIC | | | | | | | | |
| MFA- | Stable | Stable | Not | Not | — | — | — | — |
| MeNICA | | | Stable | Stable | | | | |
| MFA- | Not | Not | Not | Not | — | — | — | — |
| PCA | Stable | Stable | Stable | Stable | | | | |

Solubility

Solubility of three therapeutic agents; MFA-NIC, MFA-MeNIC and MFA-2MeNIC, which were found to be stable also underwent solubility studies. The solubility of each molecular structure was checked in distilled water as well as in normal saline. The results of the study confirmed that Mefenamic acid was found to be insoluble in water on visual inspection. No peak was observed for MFA when aqueous solution of MFA was checked in HPLC. MFA is reported to have 0.02 mg/m1 solubility.

MFA-NIC, MFA-MeNIC and MFA-2MeNIC were found to have significantly higher aqueous solubility on visual inspection than the parent MFA. Solutions of MFA-NIC, MFA-MeNIC and MFA-2MeNIC were found to have much higher solubility as compared to MFA when checked on HPLC as reported in Table 2.

Some differences were observed in the solubility of the modified MFA drug analogs when solubility in distilled water (DW) and in normal saline (NS) were compared. Denoted in Table 2 are the relatively higher solubilities of the therapeutic agent analogs when compared to the parent molecular structure, MFA.

TABLE 2
HPLC Data of MFA and stable, modified drugs of MFA

| | | | Solubility | |
|---|---|---|---|---|
| | | | in NS and | After molecular |
| | | | DW | weight correction |
| Drug | Solvent | Area | (mg/mL) | factor (mg/mL) |
| MFA Parent Drug | NS | No Peak | 0 | — |
| | DW | No Peak | 0 | — |
| MFA-NIC | NS | 2350506 | 0.10 | 0.04 |
| | DW | 6867539 | 0.28 | 0.13 |
| MFA-MeNIC | NS | 13844823 | 0.60 | 0.27 |
| | DW | 10643970 | 0.46 | 0.2 |
| MFA-2MeNIC | NS | 18274457 | 0.72 | 0.33 |
| | DW | 9876828 | 0.39 | 0.18 |

II. Additional Description

Autism and ASDs are complex diseases involving many genes along common pathways.

There is a wide spectrum of genes having mutations contributing to the risk of a child developing autism or an ASD. The identification of genetic mutations or clusters of genes associated with autism or an ASD (as described further in US2016/0206581) can be used to identify therapeutic agents to treat autism or ASD or determine whether a subject with autism or ASD will be responsive to a particular type of treatment. For example, a subject with autism or an ASD, including a subject who exhibits a mutation in an anoctamin calcium-chloride channel (for example, a mutation in an ANO1, ANO2, ANO3, ANO4, ANO5, ANO6, ANO7, ANO8, ANO9, or ANO10 channel), may be expected to be responsive to treatment with a calcium-activated chloride channel modulator, for example, a fenamate or an analog or derivative thereof. Similarly, a subject with autism or an ASD, including a subject who exhibits a mutation in a voltage-activated calcium channel (for example, a mutation in a voltage-gated calcium channel (CACNA1A . . . CACNA1S) gene), may be responsive to treatment with a voltage-activated calcium channel modulator, for example, gabapentin, pregabalin, atagabalin, or an analog or derivative thereof.

Early signs of regression toward autism or an ASD have been observed in infants as young as three months old. However, the methods of treatment described herein are not intended to be limited to use during such time period. On the contrary, the methods of the present invention can be commenced prior to such time period, for example in subjects that are newborns or infants younger than three months in age (particularly where family history or genetic testing indicates that the subject is at risk for developing autism or an ASD), and may be employed, or continued during, later stages of the life of a subject, particularly if a subject is still exhibiting indicators or symptoms of autism or an ASD. The methods of treating autism or an ASD provided by the invention include, but are not limited to, methods for preventing or delaying the onset of autism or an ASD, preventing or delaying the progression of clinical indicators or symptoms of autism or an ASD, and methods for ameliorating clinical indicators or symptoms of autism or an ASD.

The results presented herein suggest that autism and ASDs may be characterized by neuronal hyperexcitation, as are seizure disorders. In some aspects the present invention provides methods of treatment of seizure disorders, autism and ASDs that comprise administering to a subject one or more active agents that target ion channels to elicit changes in intracellular ion levels thereby causing a dampening or decrease of neuronal hyperexcitation. Such active agents may specifically target a particular class or type of ion channel or may act non-specifically on several different classes or types of ion channels to elicit a broad reduction of excitation. Furthermore, such active agents could be, for example, molecular structures or drugs that are already being used safely in humans for other indications and could be repurposed for use in the treatment of seizure disorders, autism or ASDs.

A) Active Agents

Active agents that can be used in the compositions and methods of the present invention include those that modulate the activity of ion channels, such as Cl⁻, K⁺, and/or Ca²⁺ channels, including channels that are gated or activated by a mechanism that controls the flow of ions through the channel, for example, calcium-activated, voltage-gated, or ligand-gated channels.

In some embodiments, active agents that can be used in the compositions and methods of the present invention, either alone or in combination with other active agents, include CaCC modulators. CaCC modulators are agents that cause a change in the flow or current of Cl⁻ ions through a CaCC thereby causing a change in intracellular Cl⁻. Many CaCC modulators are known in the art, and any such suitable CaCC modulator may be used in conjunction with the methods of the present invention.

In some embodiments, active agents that can be used in the compositions and methods of the present invention, either alone or in combination with other active agents, include potassium channel modulators. K⁺ channel modulators are agents that cause a change in the flow or current of of K⁺ ions through a K⁺ channel thereby causing a change in intracellular K⁺ levels. Many K⁺ channel modulators are known in the art, and any such suitable K⁺ channel modulator may be used in conjunction with the methods of the present invention.

In some embodiments, active agents that can be used in the compositions and methods of the present invention, either alone or in combination with other active agents, include Ca²⁺ channel modulators. Ca²⁺ channel modulators are agents that cause a change in the flow or current of Ca²⁺ ions through a Ca²⁺ channel thereby causing a change in intracellular Ca²⁺ levels. Many Ca²⁺ channel modulators are known in the art, and any such suitable Ca²⁺ channel modulator may be used in conjunction with the methods of the present invention. For example, gabapentin, preganalin, atagabalin, and analogs or derivatives thereof, are examples of VOCC modulators that can be used. Gabapentin is currently being used safely and effectively in human subjects, including children. Gabapentin, and/or analogs or derivatives thereof, could be repurposed for use in the compositions and methods of the present invention, for example for treatment of autism or ASDs according to the present invention. Gabapentin is indicated for several medical conditions including pediatric epilepsy (Wittkowski K M, Sonakya V, et al. (2013) Pharmacogenomics 14:391-401), and has been tested in children as young as one month old (Ouellet D, Bockbrader H N, et al. (2001) Epilepsy Res 47:229-41). In the U.S., gabapentin is currently used to treat seizures in adults and children over 3 years old. Gabapentin is available as an oral solution, tablets or capsules.

In some embodiments, active agents that can be used in the compositions and methods of the present invention, either alone or in combination with other active agents, include modulators of anoctamin calcium-activated chloride channels, for example, the ANO1, ANO2, ANO3, ANO4, ANO5, ANO6, ANO7, ANO8, ANO9, or ANO10 anoctamin calcium-activated chloride channels. A non-limiting example of a modulator of an anoctamin calcium-activated chloride channel is niflumic acid (a fenamate) which targets anoctamins to inhibit the channels. Niflumic acid is used widely (outside of the U.S.) in the treatment of joint and muscular pain. Niflumic acid, and analogs and derivatives thereof, are examples of drugs that could be repurposed for use in the compositions and methods described herein, for example those for the treatment of seizure disorders, autism or ASDs.

In some embodiments, active agents that can be used in the compositions and methods of the present invention, either alone or in combination with other active agents, include fenamates. Fenamates are a class of molecular structures that non-specifically target chloride and potassium channels, including anoctamin chloride channels and MaxK potassium channels. Potassium channels have been implicated as familial risk factors in autism and ASDs.

Derivatives of fenamates that can be used in accordance with the present invention include, but are not limited to, prodrugs of fenamates, several of which are known in the art. Such prodrugs may comprise, for example, two (or more) fenamate molecules connected directly or by a linker (or linkers) that can be degraded inside the body, such as a disulfide linker, palmityl linker, stearyl linker, glycol linker, PEG linker or ester linker. In some embodiments such prodrugs may comprise two (or more) of the same fenamate analog molecules. In some embodiments such prodrugs may comprise two (or more) different fenamate analog molecules. In some embodiments such prodrugs may comprise a fenamate analog molecule and a non-fenamate, such as a molecule of another non-fenamate active agent as described herein.

In addition to all of the classes of molecules and specific molecules described herein as potential active agents, derivatives and analogs of such molecules/agents can be used in the compositions and methods of the present invention. Derivatives of the molecules/agents described herein that can be used in accordance with the present invention include, but are not limited to, prodrug derivatives. In some embodiments such prodrugs may comprise two (or more) of the same active agent molecule, such as two or more molecules of a fenamate derivative, such as MFA-NIC In some embodiments such prodrugs may comprise two (or more) different active agent molecules, such as a molecule or a fenamate derivative (e.g. MFA-NIC) and a molecule of a calcium channel modulator (e.g. gabapentin). In some embodiments such prodrugs may comprise an active agent molecule and any another suitable molecule, moiety, or chemical group (whether an active agent or not) that can be cleaved or removed from the prodrug to release the active agent molecule.

In some embodiments the present invention provides certain novel mefenamic acid prodrug molecules, including a variety of MFA derivative analogs prodrug molecules comprising two MFA-NIC molecules joined either directly or via a linker. For example in one embodiment the present invention provides a novel MFA analog mirror prodrug having two MFA analog molecules connected by a disulfide linker, or an analog or derivative thereof. The present invention also contemplates a MFA mirror prodrug having two MFA molecules connected by PEG linker. Similarly, the present invention also provides MFA mirror prodrug molecules comprising two MFA molecules joined by a palmityl linker, stearyl linker, glycol linker, ester linker, or any other linker known in the art. The present invention also contemplates a non-mirror MFA prodrug having one MFA molecule connected to a gabapentin molecule by a PEG linker or connected by a disulfide linker, palmityl linker, stearyl linker, glycol linker, ester linker, or any other linker known in the art.

The chemical structures of the specific active agent molecules referred to by name herein are well known in the art, or are provided elsewhere in the present patent application. Similarly, methods of making such active agent molecules, and/or commercial sources from which such active agent molecules can be obtained, are well known in the art or are provided elsewhere in the present patent application.

B) Compositions and Administration

In some embodiments the present invention provides compositions comprising any one or more of the active agents described herein, either alone or in combination, for example for use in treating seizure disorders, autism or an ASD. For example, in some embodiments, the present invention provides compositions comprising a MFA analog or derivative thereof, for example for use in treating seizure disorders, autism or an ASD. In some embodiments, the present invention provides compositions comprising gabapentin, pregabalin, or atagabalin, or an analog or derivative thereof, for example for use in treating seizure disorders, autism or an ASD. In some embodiments an additional active agent may be selected from the group consisting of : ion channel modulators or an analog or derivative thereof, vitamins, such as methylated forms of Vitamin B₃, B₆, B₉, B₁₂ or an analog or derivative thereof, and where the analog or derivative of B₃ includes N,N-Dimethylnicotinamide, N-Methylnicotinamide, N-Methylnicotinic acid, or N-(1-methylethyl) carbamic acid]; and curcuminoid and curcuminoid metabolites or analogs and derivatives thereof.

In some embodiments the present invention provides compositions comprising any one of the active agents described herein (such as a fenamate analog) together with any other agent being tested for its ability to, alleviate one or more symptoms of seizure disorders, autism or an ASD, or known to, believed to, or being tested for its ability to alleviate or mitigate any one or more side-effects of the active agent (e.g. Gastrointestinal irritation in the case of fenamates such as MFA).

The compositions of pharmaceutical compositions comprising one or more active agents, as described herein, together with one or more conventionally employed components are suitable for use in pharmaceutical delivery such as carriers, stabilizers, diluents, dispersing agents, suspending agents, thickening agents, excipients, and the like, may be placed into the form of pharmaceutical formulations. Non-limiting examples of such formulations include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration. The pharmaceutical compositions and formulations of the present disclosure may, where appropriate, be conveniently presented in discrete unit dosage forms and may be prepared by any of the methods well known in the art of pharmacy. Such methods include the step of bringing into association an active agent with liquid carriers, solid matrices, semi-solid carriers, finely divided solid carriers or combinations thereof, and then, if necessary, shaping the product into the desired delivery system. Unit dosage forms of a pharmaceutical composition or formulation preferably contain a predetermined quantity of active agent and other ingredients calculated to produce a desired therapeutic effect, such as an effective amount of a therapeutically effective amount. Typical unit dosage forms include, for example, prefilled, premeasured ampules or syringes of liquid compositions, or pills, tablets, capsules, microencapsulations or the like for solid compositions.

Pharmaceutical compositions of the invention may be administered by a variety of routes including oral, buccal, sublingual, rectal, transdermal, subcutaneous, intravenous, intramuscular, intrathecal, intraperitoneal and intranasal. Depending on whether intended route of delivery is oral or parenteral, the active agents can be formulated as compositions that are, for example, either injectable, topical or oral compositions. Liquid forms of compositions may include a suitable aqueous or nonaqueous vehicle with buffers, suspending and dispensing agents, colorants, flavors and other suitable ingredients known in the art. Solid forms of compositions may include, for example, binders, excipients, lubricants, coloring agents, flavoring agents and other suitable ingredients known in the art. The active agents and pharmaceutical compositions of the invention may also be administered in sustained release forms or from sustained release drug delivery systems known in the art.

C) Dosages

The dose of an active agent of the invention may be calculated based on studies in humans or other mammals carried out to determine efficacy and/or effective amounts of the active agent. The dose amount and frequency or timing of administration may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In one embodiment of the invention, the dose of active agent is at least 0.1 mg and up to 1000 mg.

In another embodiment, the dose of active agent is in the range of 1 to 1000 mg/kg, 1 to 750 mg/kg, 1 to 500 mg/kg, 1 to 250 mg/kg, 1 to 100 mg/kg, 1 to 50 mg/kg, 1 to 25 mg/kg, 25 to 1000 mg/kg, 25 to 500 mg/kg, 25 to 100 mg/kg, 25 to 50 mg/kg, 50 to 1000 mg/kg, 50 to 500 mg/kg, or 50 to 100 mg/kg. In some embodiments the above dosages are mg/kg/day.

In one embodiment, a single dose may be administered. In another embodiment, multiple doses may be administered over a period of time, for example, at specified intervals, such as, four times per day, twice per day, once a day, weekly, monthly, and the like.

Mefenamic acid and gabapentin are presently used in children and have established dosing regimens. In some embodiments such established dosing regimens may be used in conjunction with the treatment methods of the present invention. For example, currently, infants and children over 6 months old are given mefenamic acid at a dose of 25 mg/kg/day, which may be in several divided doses, for example 3 divided doses a day of 50 mg for children 6 months to under 2 years, 100 mg dose for children 2 years to under 5 years, 150 mg dose for children 5 years to under 9 years, and 200 mg dose for children 9 years to 12 years, for the treatment of pain and/or fever (see Public Assessment Report for pediatric studies submitted in accordance with Article 45 of Regulation (EC) No1901/2006, as amended; Mefenamic Acid; UK/W/037/pdWS/001 (17 Sep. 2012), the contents of which are hereby incorporated by reference. Such doses of MFA can be used in accordance with the methods of the disclosure for treatment of seizure disorders, autism or ASDs, or can be used as starting points or guides in the performance of studies, such as dose-response and/or dose-escalation studies, aimed at determining effective amounts of MFA, or other fenamates, or analogs or derivatives thereof, to be used in accordance with the methods of the present disclosure for treatment of seizure disorders, autism or ASDs.

Similarly, dosing regimens for gabapentin for the treatment of epileptic seizures have been established in children and adults. For example, in pediatric patients age 3-12 years, the starting dose ranges from 10-15 mg/kg/day in three divided doses, and the effective dose is subsequently reached by upward titration over a period of approximately 3 days. The effective dose is determined according to the age of the child, as follows: 25-35 mg/kg/day for children over 5 years of age and 40 mg/kg/day for children ages 3-4 years. For the treatment of epileptic seizures in patients over 12 years of age, the starting dose of gabapentin is 300 mg, three times a day, and the effective dose is 900 to 1800 mg/day in divided doses (three times a day) using 300 or 400 mg capsules, or 600 to 800 mg tablets. Such doses of gapapentin can be used in accordance with the methods of the disclosure for treatment of autism or ASDs, or can be used as starting points or guides in the performance of studies, such as dose-response and/or dose-escalation studies, aimed at determining effective amounts of gabapentin, or analogs or derivatives thereof, to be used in accordance with the methods of the disclosure for treatment of autism or ASDs.

In embodiments where two (or more) active agents are to be used in a method of treatment, whether those active agents are present in separate compositions, the same composition, or even the same molecule (as in the case of some of the prodrugs described herein), a lower dosage of each active agent may be used than would be used if the active agents were used alone. For example, in embodiments where a fenamate, such as MFA or an analog or derivative thereof, is to be used in combination with another active agent, such as gabapentin, or an analog or derivative thereof, the dose used for each may be reduced as compared to the sample dosages set forth above. For example, in some embodiments, where two active agents are to be used, the dose of each active agent used may be half of that used for the single agents.

Subjects

The methods and compositions provided by the invention may be used to treat a neurodegenerative disorder, seizure disorder, and or neurodevelopmental disease or disorder, such as autism and/or an ASD, in any subject in need of such treatment. In one embodiment, the subject is a human. It should be noted that, while in some embodiments the subjects to be treated are young children of around 3 months to 3 years in age, in other embodiments the methods of treatment described herein are not intended to be limited to such subjects. Rather, in some embodiments the subjects can be of any age, ranging from newborns to older adults. In some embodiments it may be desirable to treat very young subjects, for example newborns and/or young infants, particularly where family history or genetic testing indicates that the subject is at risk for developing seizure disorders, autism or an ASD. Similarly, in some embodiments it may be desirable to treat much older subjects, particularly where such subjects are still exhibiting indicators or symptoms of seizure disorders, autism or an ASD.

The methods and compositions of the invention may be employed as prophylactic treatments or therapeutic treatments. For prophylactic treatments, the methods and compositions provided herein can be used preventatively in subjects that do not yet exhibit any clear, definitive, or detectable clinical indicators or symptoms of the disease or disorder but that are believed to be at risk of developing the disease or disorder, such as seizure disorders, autism or an ASD. A subject receiving prophylactic treatment for seizure disorders, autism or an ASD, for example, may not exhibit any clinical indicators or symptoms of seizure disorders, autism or an ASD. In the case of therapeutic treatments, the methods and compositions provided herein can be used in subjects that already exhibit one or more clinical indicators or symptoms of the disease or disorder, such as seizure disorders, autism or an ASD. A subject receiving therapeutic treatment for seizure disorders, autism or an ASD, for example, may have been clinically diagnosed with a seizure disorder, autism or an ASD or may otherwise exhibit one or more clinical indicators or symptoms of a seizure disorder, autism or an ASD.

In one embodiment of the invention, a subject may have been identified as being at risk of developing a seizure disorder, autism or an ASD. In one embodiment, the subject has a family history of seizure disorders, autism or an ASD. In one embodiment, the subject has one or more genetic risk factors associated with a seizure disorder, autism and/or ASDs, for example, a genetic mutation in a gene encoding a chloride channel, potassium channel, and/or a calcium channel.

In some embodiments, a subject to be treated using the methods and/or compositions of the present invention is a newborn human, or a human child of at least 1 day in age, 1 week in age, 1 month in age, 2 months in age, 3 months in age, 4 months in age, 5 months in age, 6 months in age, 7 months in age, 8 months in age, 9 months in age, 10 months in age, 11 months in age, 12 months in age, 13 months in age, 14 months in age, 15 months in age, 16 months in age, 17 months in age, 18 months in age, 19 months in age, 20 months in age, 21 months in age, 22 months in age, 23 months in age, 24 months in age, or 36 months in age. In one embodiment, the subject is a human child of less than 36 months in age, 30 months in age, 24 months in age, 18 months in age, 17 months in age, 16 months in age, 15 months in age, 14 months in age, 13 months in age, 12 months in age, 11 months in age, 10 months in age, 9 months in age, 8 months in age, 7 months in age, 6 months in age, 5 months in age, 4 months in age, or 3 months in age. In one embodiment, the subject is a human child of between 3 months and 36 months in age, 3 months and 24 months in age, 3 months and 18 months in age, 3 months and 12 months in age, 6 months and 36 months in age, 6 months and 24 months in age, 6 months and 18 months in age, 6 months and 12 months in age, 9 months and 36 months in age, 9 months and 24 months in age, 9 months and 18 months in age, 9 months and 12 months in age, 12 months and 36 months in age, 12 months and 24 months in age, or 12 months and 18 months in age. In one embodiment, the subject is a human child of 3 months in age, 4 months in age, 5 months in age, 6 months in age, 7 months in age, 8 months in age, 9 months in age, 10 months in age, 11 months in age, 12 months in age, 13 months in age, 14 months in age, 15 months in age, 16 months in age, 17 months in age, 18 months in age, 19 months in age, 20 months in age, 21 months in age, 22 months in age, 23 months in age, 24 months in age, 25 months in age, 26 months in age, 27 months in age, 28 months in age, 29 months in age, 30 months in age, 31 months in age, 32 months in age, 33 months in age, 34 months in age, 35 months in age, or 36 months in age.

Pre-Clinical Outcomes

In some embodiments the methods of treatment provided herein (which comprise, for example, administering to a subject an effective amount of a composition according to the present invention) result in, or are aimed at achieving, a detectable improvement in permeability of the BBB and alteration in brain metabolites.

Structure (I), MFA-NIC, was assessed as a possible therapeutic in a rat seizure pre-clinical model. Assessments were conducted using Contrast-enhanced (CE) magnetic resonance (MR) imaging (MRI) and MR spectroscopy (MRS). The seizure model initially involved injection of pilocarpine (380 mg/kg i.p.) alone (Ikonomidou-Turski et al., 1988), however all rats undergoing this procedure died before MRS data could be obtained. A more recent model by Jeong et al. (2017) was adopted, where a lower dose of pilocarpine (25 mg/kg i.p.) was preceded by lithium chloride (127 mg/kg i.p.) 19 hours before, and scopolamine (2 mg/kg i.p.) 30 min. before, pilocarpine. Injection of pilocarpine induced status epilepticus about 20-30 min post-pilocarpine (Jeong et al., 2017). Pascente et al. (2016) also used lithium chloride 18 hours before pilocarpine. Scopolamine was also used by Behr et al. (2017) 30 m in before pilocarpine. Electroencephalography (EEG) was used in the Behr et al. study to confirm seizures.

A MRI contrast agent, Gd-DTPA (gadolinium diethylene triamine penta acetic acid) was used to establish if there was increased BBB permeability. Gd-DTPA does not cross the BBB in a normal brain with an intact BBB. Three brain regions were assessed: hippocampus, thalamus and cerebral cortex. In this study, CE-MRI indicated that although the seizure group had a slightly higher change in MRI signal intensity, this group was not significantly different than the saline-treated control group. This may be due to the variability in the change in MRI signal intensity in the seizure group, and the low number of rats per group. The smaller variability in the saline-treated controls was due to the large animal size of n=25 (obtained from a previous study conducted in the Towner group). The normal untreated controls (n=5) also had a larger variability than the saline-treated controls. The saline-treated controls were used for all statistical comparisons.

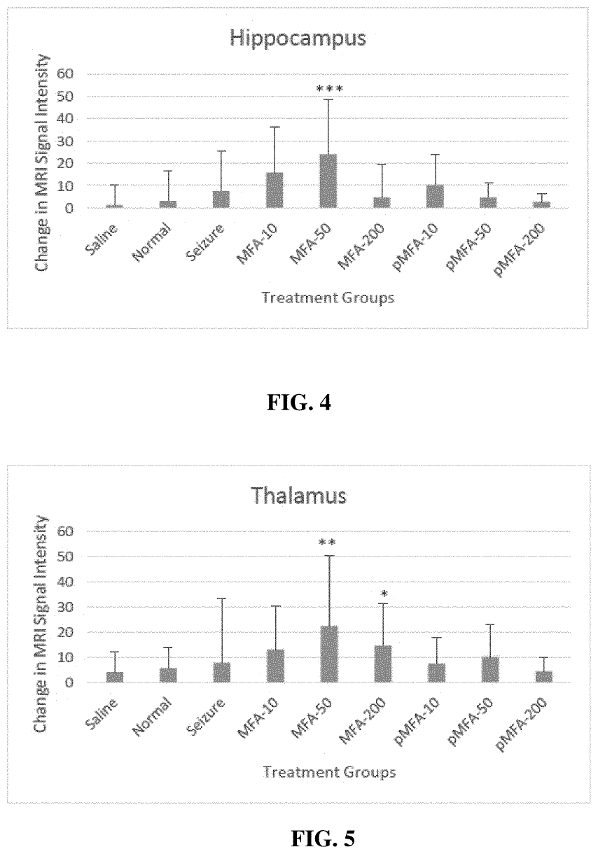

In the hippocampus (see FIG. 4), it was found that only the MFA 50 mg/kg dose had a significant increase in BBB permeability. None of the MFA-NIC (shown in FIGS. 4-11 as pro-MFA or pMFA)-treated rats were found to be significantly different than saline-treated controls.

In the thalamus (see FIG. 5), it was found that both the MFA 50 mg/kg and MFA 200 mg/kg dose groups had a significant increase in BBB permeability. None of the MFA-NIC (pro-MFA or pMFA)-treated rats were found to be significantly different than saline-treated controls.

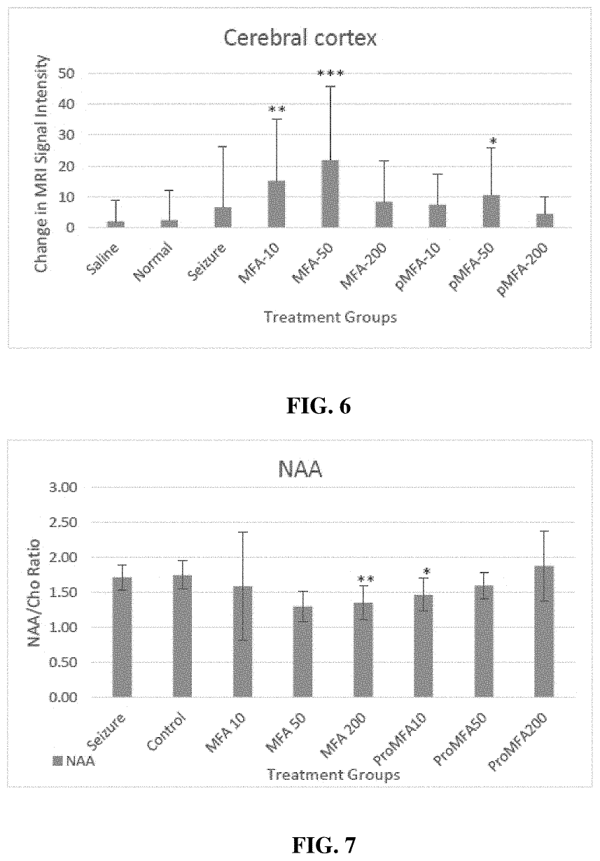

In the cerebral cortex (see FIG. 6), it was found that both the MFA 50 mg/kg and MFA 200 mg/kg, as well as the pro-MFA 50 mg/kg, dose groups had a significant increase in BBB permeability. The MFA-NIC (pro-MFA or pMFA)-treated rats treated with 10 or 200 mg/kg doses were found to be not significantly different than saline-treated controls.

Regarding BBB permeability, it can be said that pro-MFA (MFA-NIC), when administered to pilocarpine-induced seizure rats, was found to be similar to saline controls in most cases (minor exception of increased BBB permeability for the 50 mg/kg dose in the cerebral cortex). The 200 mg/kg dose for MFA-NIC was found to be the most effective in essentially restoring any seizure effects back to those in the saline-treated controls, i.e. MFA-NIC treatment at this dose was effective in preventing an increase in BBB permeability.

MRS was used to assess levels of brain metabolites, including N-acetyl aspartate (NAA), total creatine (Cr, total choline (Cho), taurine (Tau), glutamate (Glu) and myo-inositol (Myo-Ins). Cho was used as a normalization reference for all other metabolites. For the control group a total of 10 rats were assessed. For all other treatment groups n=5.

NAA/Cho ratios (FIG. 7) indicated that there was a significant decrease for the 200 mg/kg MFA and 10 mg/kg pro-MFA (MFA-NIC) treatment groups compared to the control (non-treated, non-seizure) group. There was no significance in NAA/Cho ratios between the control and pilocarpine-induced seizure groups. Perhaps the extent of the seizures was not severe enough to decrease NAA.

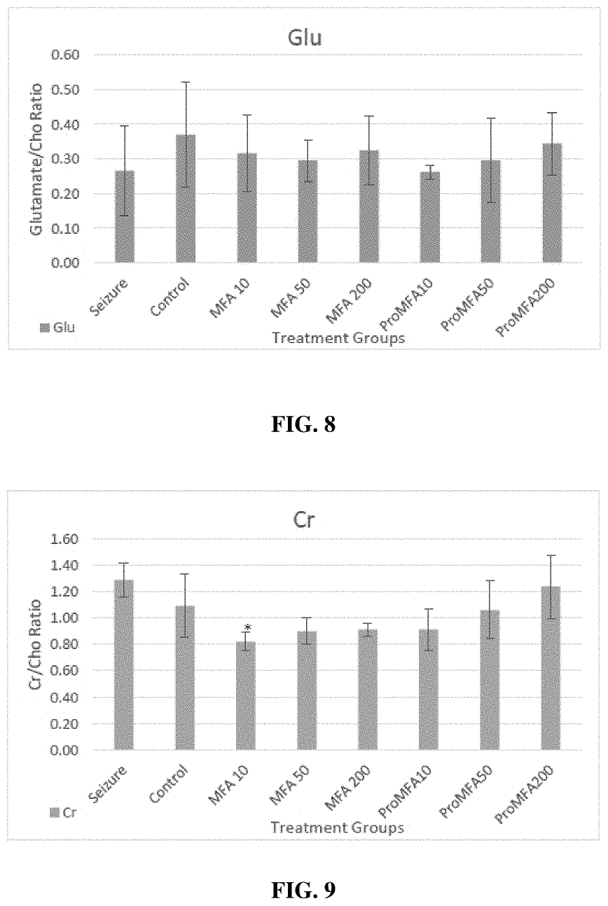

Glu/Cho ratios (FIG. 8) indicated that there were no significant changes for any of the treatment groups compared to the control (non-treated, non-seizure) group. Although the levels of Glu seem to decrease in the seizure group compared to the control group, this was not found to be significantly different. This may be due to the variability in both of these groups.

Cr/Cho ratios (FIG. 9) indicated that there was a significant decrease for the 10 mg/kg MFA treatment group compared to the control (non-treated, non-seizure) group. Although Cr levels seem to increase in the seizure animals compared to controls, there was no significance in Cr/Cho ratios between these two groups.

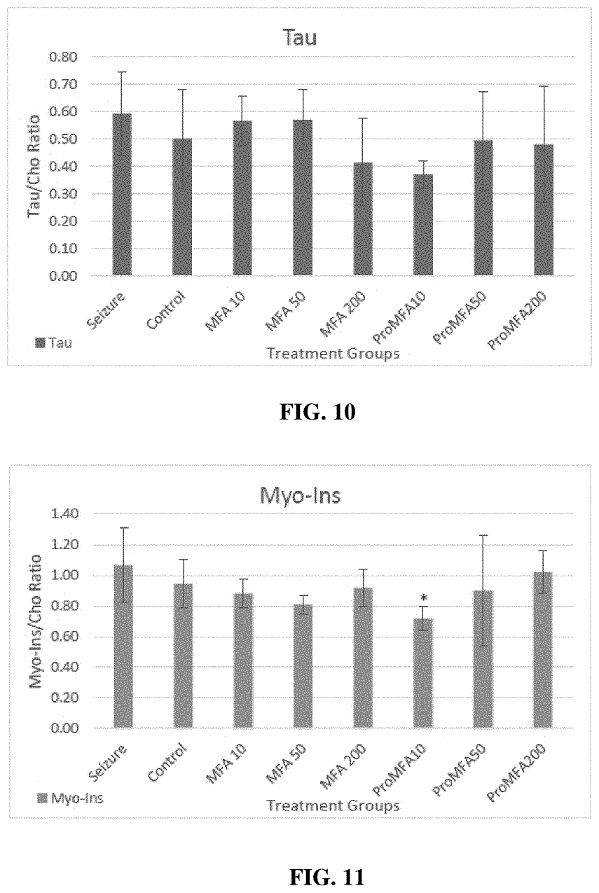

Tau/Cho ratios (FIG. 10) indicated that there were no significant changes for any of the treatment groups compared to the control (non-treated, non-seizure) group. Although the levels of Tau seem to increase in the seizure group compared to the control group, this was not found to be significantly different. This may be due to the variability in both of these groups.

Myo-Ins/Cho ratios (FIG. 11) indicated that there was a significant decrease for the 10 mg/kg pro-MFA (MFA-NIC) treatment group compared to the control (non-treated, non-seizure) group. There was no significance in Myo-Ins/Cho ratios between the control and seizure groups.

Regarding brain metabolite alterations, it can be said that pro-MFA (MFA-NIC), when administered to pilocarpine-induced seizure rats, was found to be similar to controls in most cases (minor exception of decreased Myo-Ins/Cho ratio for the 10 mg/kg dose). The 200 mg/kg dose for MFA-NIC was found to be the most effective in essentially restoring any seizure effects back to those levels found for controls, i.e. MFA-NIC treatment at this dose was effective in restoring brain metabolite levels back to normal.

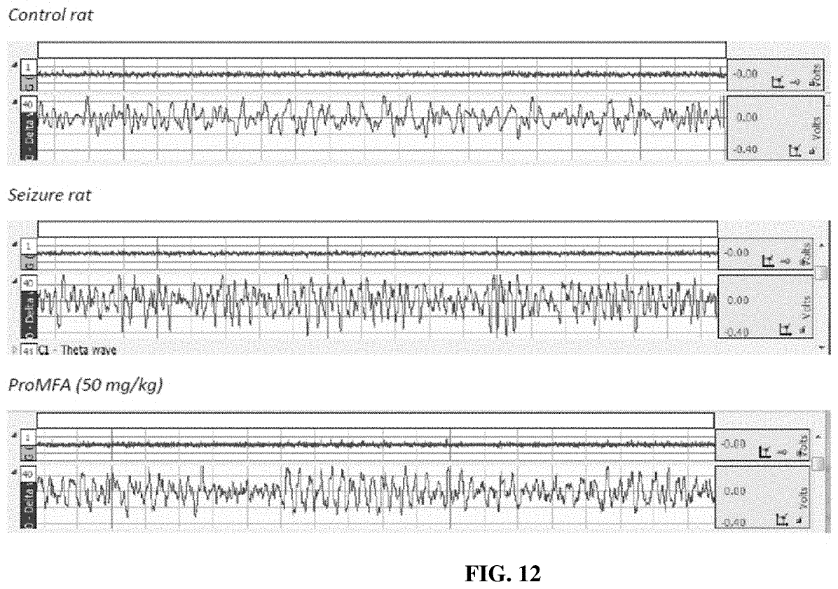

EEG (see FIG. 12) shows that the seizure model worked, where seizure episodes were detected (compared to normal controls). Preliminary data indicated that pro-MFA (MFA-NIC) does reduce the level of seizure episodes.

By analyzing the EEG patterns, neurologists can identify seizures and other altered brain activity of concern. Often patients who have autism need EEG protocols that address their sensory and communication challenges.

In some embodiments the methods of the present disclosure may be initiated in very young human subjects, for example from birth up to approximately 36 months of age. Onset of early signs of regression towards autism or ASDs occurs gradually, and the methods provided by this disclosure can be employed when early symptoms are detected. Some evidence suggests that neuronal growth stops at around two years of age, therefore, in some embodiments, the methods of treatment provided herein are initiated in infants and children prior to or around 2 years of age, during the active phase of neuronal growth and development in the brain.

Most autism and ASD behavioral symptoms emerge during the second year of life, before a clinical diagnosis can be made. In young subjects where behavioral symptoms are not evident or detectable, other measureable indicators are needed to assess early signs of regression toward autism or ASDs. Examples of such methods include, behavioral evaluation, eye tracking, skin conductance/galvanic skin response, and EEG, all of which can measure underlying brain function in young subjects who do not display definitive behavioral symptoms of autism or ASDs. These methods can be used to measure changes in brain activity in response to stimuli, for example, visual stimuli such as familiar vs. unfamiliar faces; direct vs. averted eye gaze; or still face vs. speaking.

Electroencephalography (EEG) can be used to measure brain activity in response to the presentation of stimuli and can detect alterations in brain activity which may indicate subtle brain function abnormalities before behavioral symptoms are apparent. Decreased complexity of an EEG signal indicates abnormal brain connectivity and has been used as a biomarker to determine ASD risk (Bosl W, Tierney A, et al. (2011) BMC Medicine 9:18). Magnetoencephalography (MEG) (Yoshimura Y, Kikuchi M, et al. (2013) PLoS One 8:e80126) may also be used to measure brain activity.

The above methods are known in the art and can be implemented into dose-response trials to measure efficacy and determine effective amounts of active agents provided by the invention to treat seizure disorders, autism or ASDs.

The compositions and methods described herein are illustrative only and are not intended to be limiting. Those of skill in the art will appreciate that various combinations or modifications of the specific compositions and methods described above can be made, and all such combinations and modifications of the compositions and methods described herein may be used in carrying out the disclosure.

Examples of Therapeutic Techniques for Utilizing MFA Analogs and/or Derivatives

Few drugs, however, are known to target individual anoctamins or even exclusively CaCCs. Cl⁻ channel blockers such as fenamates, for example, may decrease neuronal excitability by activating Ca²⁺-dependent outward rectifying K⁺ channels.

Drugs that target ion channels may decrease hyperexcitation to a level where a child does not feel the need to withdraw from social interaction. Gabapentin, approved by the FDA for the treatment of partial seizures in children from three years of age (Parke-Davis (2013) Medication Guide Neurontin. New York, N.Y., Pfizer: 32-7) and tested in population-pharmacokinetic studies including subjects starting from age one month (Ouellet D, Bockbrader F I N, et al. (2001) Epilepsy Res 47:229-41), might be repurposed for children with autism or ASDs, such as those with mutations in VOCCs. Similarly, drugs targeting the CaCCs identified herein, such as fenamates, which have so far been considered in pain, in general, and (menstrual) migraines (Pringsheim T, Davenport W J, et al. (2008) Neurology 70:1555-63) in particular, as well as in epilepsies for decreasing excitatory synaptic activity and reducing neuronal excitability (Fernandez M, Lao-Peregrin C, et al. (2010) Epilepsia 51:384-90; Yau H J, Baranauskas G, et al. (2010) J Physiol 588:3869-82), might also be repurposed for treatment of autism or ASDs, for example in subjects with mutations involving CV signaling. Fenamates target a variety of potassium and chloride channels (Greenwood I A, Leblanc N (2007) Trends Pharmacol Sci 28:1-5) and, thus, may have less systemic side effects (including hypokalaemia (Ng T M, Konopka E, et al. (2013) J Cardiovasc Pharmacol Ther 18:345-53)) than the drugs that inhibit Cl⁻ influx via the Na⁺—K⁺-2Cl⁻ co-transporter NKCC1, which have been shown to improve symptoms of ASD in some cases (Lemonnier E, Degrez C, et al. (2012) Transl Psychiatry 2:e202).

The overlap in genetic risk factors between ASD and CAE suggests another potential benefit of the proposed early intervention. As neonatal seizures per se may cause long-term neurological problems (Nardou R, Ferrari D C, et al. (2013) Semin Fetal Neonatal Med 18:175-84), preventing the postulated intolerable experiences may positively affect a wider range of ASD symptoms.

Excessive neuronal growth through impaired control of growth signaling may explain the larger brain and body sizes seen in children with severe forms of autism (Pathan A R, Karwa M, et al. (2010) Inflammopharmacol 18:157-68), and, importantly, may leave neurons overly sensitive to stimulation via the second messenger Ca²⁺. Prior research suggests that pharmaceutical interventions targeting excitatory signaling should be started in very early childhood when rapid neural development is occurring, for example from 6-12 months of age, the same time where language regression is seen in some children at the beginning of the ‘stranger anxiety’ period, or even earlier.

For example, according to the present invention, gabapentin, approved by the FDA for the treatment of partial seizures in children from 3 years of age (Parke-Davis (2013) Medication Guide Neurontin. New York, N.Y., Pfizer: 32-7) and tested in population-pharmacokinetic studies included subjects starting from one month of age (Ouellet D, Bockbrader H N, et al. (2001) Epilepsy Res 47:229-41), can be repurposed for children at risk of developing autism or an ASD, or exhibiting one or more indicators or symptoms of autism or an ASD. For example, such children may have mutations in VOCCs. Similarly, since the results described in Example 1 herein suggest that mutations involving K⁺ and Cl⁻ signaling may be associated with ASD, fenamates, which have so far been used or considered for treatment of pain, including (menstrual) migraines (Pringsheim T, Davenport W J, et al. (2008) Neurology 70:1555-63), as well as epilepsies for decreasing excitatory synaptic activity and reducing neuronal excitability (Fernandez M, Lao-Peregrin C, et al. (2010) Epilepsia 51:384-90; Yau H J, Baranauskas G, et al. (2010) J Physiol 588:3869-82), can also be repurposed for treatment or prevention of autism or ASD. The fenamate mefenamic acid (MFA) has been used in preterm children (Ito K, Niida Y, et al. (1994) Acta Paediatr Jpn 36:387-91) and, in the UK, is recommended for use in infants starting at six months of age (Heads of Medicines Agencies UK (2012)).

MFA analogs and derivatives thereof (either alone or in combination) or a placebo can be administered to children at high risk of developing seizure disorders, autism or ASD, such as male siblings of children with ASD, starting at the time where first symptoms of regression are observed, which is expected to be around the age of 12 months, or earlier.

The ion channel modulators described herein have been used safely in children for decades to treat, e.g., arthritis and epilepsies.

MFA was introduced in the early 1960s as an NSAID and has been approved in the U.K. for the treatment of juvenile arthritis from 6 months (Heads of Medicines Agencies UK (2012)). Children with juvenile idiopathic arthritis (JIA) have been treated chronically without raising any safety concerns. Hence, doses, biological activity, tolerability, and feasibility of administering a MFA oral suspension in the at risk population are well establishedln the U.S., MFA is approved for the treatment of pain and menstrual migraines, which have a poor response to analgesics (Pringsheim T, Davenport W J, et al. (2008) Neurology 70:1555-63). Only in the 1990s was it discovered that MFA exerts part, if not most, of its effect by reducing neuronal excitability through opening K⁺ and modulating Cl⁻ channels. (Peretz A, Degani N, et al. (2005) Mol Pharmacol 67:1053-66; Peretz A, Degani-Katzav N, et al. (2007) PLoS One 2:e1332)

Prodrugs of mefenamic acid (MFA) have been shown to decrease the most frequent adverse events associated with MFA, such as transient GI disturbances. (Shah K, Shrivastava S K, et al. (2014) Pak J Pharm Sci 27:917-23).

Mefenamic Acid Analog Prodrug

A fenamate prodrug, specifically a MFA analog prodrugs can be linked via a linkage at the carboxyl moiety, to reduce gastrointestinal (GI) side effects, to slow release, to lower peak concentration in favor of more stable blood levels, and to reduce the risk of accidental overdose. (Prodrugs of NSAIDS have a “more favorable therapeutic ratio of anti-inflammatory and gastrointestinal erosive activities”. (Venuti M C, Young J M, et al. (1989) Pharm Res 6:867-73)). MFA is available in the U.S. as Ponstel (in pill form for adults) and in the U.K. as Ponstan (also as an oral suspension for children from 6 mo of age). MFA (UNII: 367589PJ2C) is a nonsteroidal anti-inflammatory drug (NSAID), but with weak COX inhibition and high potency for opening potassium channels.

The MFA prodrug analogs can be tested against placebo and parent MFA in a randomized, double-blind trial

Synthesis: Fenamates are N-phenyl-substituted anthranilic acid derivatives comprising a class of molecules based on fenamic acid (2-(phenylamino)benzoic acid. MFA can be synthesized by the copper-catalyzed condensation of o-chlorobenzoic acid and 2,3,dimethylaniline (see (Kurali (2012) CN101704761B) which describes synthesis of MFA from o-chlorobenzoic acid and 2,3 ,dimethylaniline (CN101704761B)).

Properties: MFA is a white to grayish-white, odorless, microcrystalline powder with a melting point of 230-231° C. with effervescence (corrected) and water solubility of 0.004% at pH 7.1. The molecular weight is 241.3 and the pKa (in water) is 4.2. (Winder C V, Wax J, et al. (1962) J Pharmacol Exp Ther 138:405-13)

There is a paucity of animal models of autism and ASD′. Most animal models for autism model ‘syndromic’ forms of autism, where a single genetic variation causes an ‘autism-like’ phenotype. There is a lack of animal models for idiopathic autism. Because the methods and compositions of the present invention are postulated to involve, in part, broadly reducing neuronal excitability, animal models of neuronal excitability, in general, could be drawn on as models for idiopathic forms of autism. For example, fenamates, in general, and MFA, in particular, have shown efficacy in animal models of seizures. Accordingly, in some embodiments such seizure models have been used in conjunction with the novel compositions and methods described herein.

The properties of several MFA (and related) prodrugs have been studied. In a study of several NSAID esters and thioesters (including MFA), “each prodrug retained the anti-inflammatory activity characteristic of the corresponding parent drug but exhibited moderately to greatly reduced GI erosive properties and significantly reduced analgetic potencies.” (Venuti M C, Young J M, et al. (1989) Pharm Res 6:867-73) A separate study on an MFA prodrug confirmed the high stability. (Jilani J A, Pillai G K, et al. (1997) Drug Dev Ind Pharm 23:319-23)

Pharmacokinetics and Product Metabolism of MFA in Humans

Because both renal and hepatic excretion are involved in elimination, dosage adjustments in patients with renal/hepatic dysfunction may be necessary. Given that MFA, its metabolites and conjugates are primarily excreted by the kidneys, the potential exists for MFA metabolites to accumulate in patients with preexisting renal disease or significantly impaired renal function. As hepatic metabolism is also a significant pathway of MFA elimination, patients with acute and chronic hepatic disease may require reduced doses. Lacking most of the above risk factors, infants tolerate NSAIDs well. GI symptoms appear to be less common than in adults and renal toxicity is rare. (Hollingworth P (1993) Br J Rheumatol 32:73-7)

Pediatric use: Lacking most of the above risk factors, infants tolerate NSAIDs well. GI symptoms appear to be less common than in adults and renal toxicity is rare. (Hollingworth P (1993) Br J Rheumatol 32:73-7). Children taking corticosteroids, anticoagulants, or APAP/NSAIDs, as well as children in poor health, including those having heart disease, hepatic and/or renal dysfunction, should be excluded from inclusion in trials as determined by a physician until further studies can be completed.

In the U.K., MFA is licensed as an anti-inflammatory analgesic for the symptomatic relief of rheumatoid arthritis (RA), osteoarthrosis as well as pain and pyrexia. It is also licensed for primary dysmenorrhea in adolescents and menorrhagia. In addition to 250 capsules, MFA is also available as 500 mg tablets and as a 50 mg/5 ml oral suspension

The UK Public Assessment Report: (Heads of Medicines Agencies UK (2012)) concludes: the “overall the safety profile of [MFA] in the paediatric population does not appear to be different from adults.”

Approved indications for MFA: In the U.S., MFA was approved in 1967 (NDA #015034) and is available as a prescription drug (a) for relief of mild to moderate pain in patients ≥14 yr and (b) for treatment of primary dysmenorrhea (U.S. Food and Drug Administration (2008)) in capsules of 250 and 500 mg, in generic forms and under the brand name Ponstel® (Shinogi Inc.). The recommended dose is 250-500 mg 3-4 times daily for <7 days. In the U.K., MFA is licensed as an NSAID for the symptomatic relief of rheumatoid arthritis (RA), osteoarthrosis as well as pain and pyrexia. It is also licensed for primary dysmenorrhea in adolescents and menorrhagia. MFA is available in tablets, capsules and as an oral suspension for children [6 months-12 years] to be given at a dose of 25 mg/kg of bodyweight in divided doses. Duration should be ≤7 days, except for Still's Disease (JIA). (Heads of Medicines Agencies UK (2012))

The present disclosure provides compositions and methods that may be useful for treating subjects showing early indicators or symptoms of seizure disorders, autism or an ASD, or subjects that are at risk of developing autism or an ASD. In some embodiments, treatment with a fenamate, such as a MFA analog, or a prodrug thereof, is provided. Without wishing to be bound by theory, it is hypothesized that such treatments may reduce neuronal over-excitation during critical developmental periods and, thereby, avoid withdrawal from verbal and social interactions, which, over time, could cause experience-dependent pruning in functionally related cortical regions.

A MFA ester prodrug was shown to have high bioavailability, albeit with a slower pharmacokinetics. (Jilani J A, Pillai G K, et al. (1997) Drug Dev Ind Pharm 23:319-23) Hence, the established safe dose of 25 mg/kg/d (Heads of Medicines Agencies UK (2012)) (b.i.d.) can be used in this trial.

For the purposes of those jurisdictions that permit incorporation by reference, the text of all documents cited herein are hereby incorporated by reference in its entirety.

In compliance with the patent laws, the subject matter disclosed herein has been described in language more or less specific as to structural and methodical features. However, the scope of protection sought is to be limited only by the following claims, given their broadest possible interpretations. The claims are not to be limited by the specific features shown and described, as the description above only discloses example embodiments. While the foregoing is directed to preferred embodiments of the present invention, other and further embodiments of the invention may be devised without departing from the basic scope thereof, and the scope thereof is determined by the claims which follow.

In a further configuration or implementation of the methods and compositions described herein, in one particular implementation or embodiment, one or more mefenamic acid (MFA) analog molecules are provided comprising one or more molecular structures of mefenamic acid, specifically molecular structures IX or X;

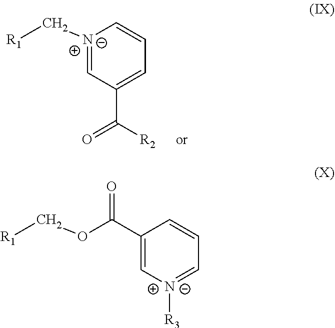

wherein R₁ is selected from the group consisting of anthranilic acid esters, including fenamic acid, MFA, tolfenamic acid (TFA), flufenamic acid (FFA), meclofenamic acid (CFA), and analogs and derivatives thereof;

R₂ is selected from the group consisting of an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, alanine, and lysine or derivatives thereof;

and R₃ is selected from the group consisting of a lower alkyl, an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, N-(1-methylethyl) carbamic acid, alanine, and lysine or derivatives thereof;

In a further configuration or implementation of the methods and compositions described herein, in one particular implementation or embodiment, the one or more analog molecules of MFA of molecular structure (IX) of the previously described implementations where R₁ is MFA and R₂ is —NH2, that comprises an MFA-Nicotinamide molecule (MFA-NIC), molecular structure (XI);

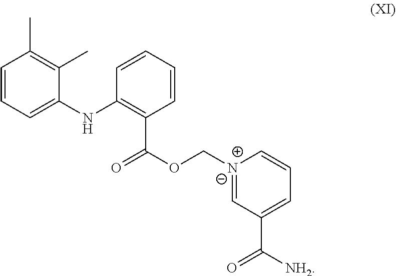

In a further configuration, the one or more analog molecules of MFA of molecular Structure (IX)of the previously described implementations, wherein R₁ is MFA and R₂ is —N(CH₃)₂ that comprises a MFA- N,N-Dimethylnicotinamide N-Methylnicotinamide (MFA-2MeNIC) molecular structure (XII);

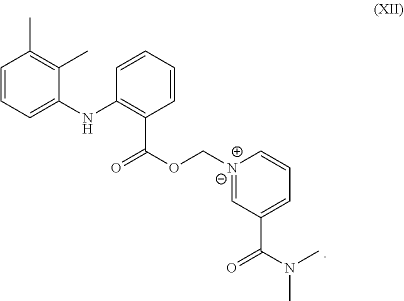

In a further configuration, the one or more analog molecules of MFA of Structure (IX) of the previously described implementations, wherein R₁ is MFA and R₂ is —NHCH₃, that comprises a MFA- N-Methylnicotinamide (MFA-MeNIC) molecular structure (XIII);

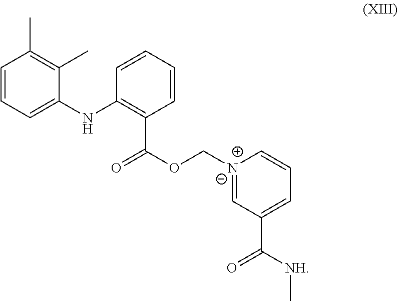

In a further configuration, the one or more analog molecules of MFA of molecular Structure (X) of the previously described implementations, wherein R₁ is MFA and R₃ is —CH₃, that comprises a MFA-N-methylnicotinic acid (MFA-MeNICA) molecular structure (XIV);

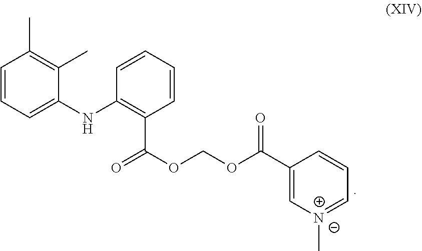

In a further configuration, the one or more analog molecular Structures (X) of the previously described implementations, wherein R₁=MFA and R₃=N-(1-methylethyl) carbamic acid, MFA-Pyridinium,3-carboxy-1-[4N-(1-methylethyl) carbamic acid] (MFA-PCA) molecular structure (XV).

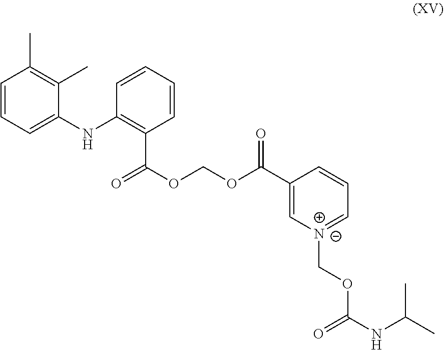 [SMILES: Cc1cccc(Nc2ccccc2C(=O)OCOC(=O)c2ccc[n+](COC(=O)NC(C)C)c2)c1C]

In a further configuration, the one or more mefenamic acid (MFA) analog molecular structures of any of the preceding implementations, wherein said molecular structures provide a basis for compositions comprising both a fenamate and analogs or derivatives thereof, including a calcium channel modulator consisting of a group of analogs or derivatives selected from one or more of gabapentin, pregabalin, and atagabalin.

In a further configuration, the one or more mefenamic acid (MFA) analog molecular structures of the previously described implementations, wherein said fenamate analogs or derivative thereof are selected from one or more of a group of molecular structures consisting of MFA-NIC (XI), MFA-MeNIC (XII), MFA-2MeNIC (XIII), MFA-MeNICA (XIV) and MFA-PCA(XV).

In a further configuration, the one or more mefenamic acid (MFA) analog molecular structures of any of the preceding implementations, wherein analog molecular structures MFA-NIC (XI), MFA-MeNIC (XII), and MFA-2MeNIC (XIII), exhibit solubility when immersed in distilled water and acetonitrile.

In a further configuration, the one or more mefenamic acid (MFA) analog molecular structures of the previously described implementations, wherein analog molecular structures MFA-NIC (XI), MFA-MeNIC (XII), and MFA-2MeNIC (XIII), exhibit stability when immersed in distilled water, saline solution, and acetonitrile.

In a further configuration, the one or more mefenamic acid (MFA) analog molecular structures of any of the preceding implementations, wherein analog molecular structures MFA-MeNICA (XIV) and MFA-MeNICA (XV) exhibit stability when immersed in acetonitrile.

In a further configuration, the one or more mefenamic acid (MFA) analog molecular structures of the previously described implementations, wherein analog molecular structure MFA-MeNICA (XV) exhibits stability when immersed in distilled water and acetonitrile.

In a further configuration, a method of improving one or more indicators or symptoms of autism or an autism spectrum disorder (ASD) in a human subject is provided wherein the method comprises administering to a subject exhibiting one or more indicators or symptoms of autism or an ASD, or a subject at risk of developing autism or an ASD, an effective amount of one or more analog molecular structures of MFA, wherein said analog molecular structures IX or X are;

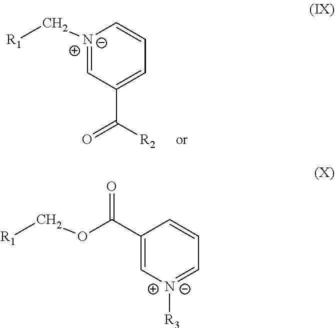

wherein R₁ is selected from the group consisting of anthranilic acid esters, including fenamic acid, MFA, tolfenamic acid (TFA), flufenamic acid (FFA), meclofenamic acid (CFA), and analogs and derivatives thereof;

R₂ is selected from the group consisting of an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine, 2,2′-oxybis ethanamine, alanine, and lysine or derivatives thereof;

and R₃ is selected from the group consisting of a lower alkyl, an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, N-(1-methylethyl) carbamic acid, alanine, and lysine or derivatives thereof;

In a further configuration, the method of the previously described implementation, wherein said one or more analog molecular structures is an analog to Structure (IX), wherein R₁ is MFA and R₂ is —NH₂, that further comprises an MFA-Nicotinamide molecule (MFA-NIC), resulting in molecular structure (XI);

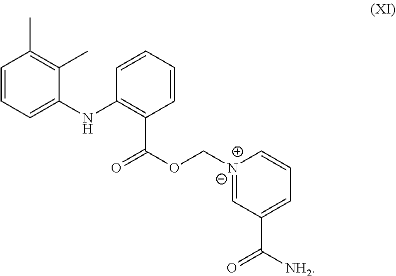

In a further configuration, he method of the previously described implementations, wherein said one or more analog molecular structures is an analog to Structure (IX), wherein R₁ is MFA and R₂ is —N(CH₃)₂ that further comprises a MFA- N,N-Dimethylnicotinamide N-Methylnicotinamide (MFA-2MeNIC) resulting in molecular structure (XII);

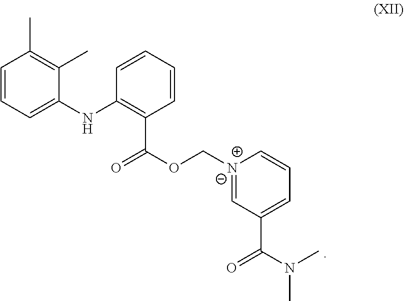

In a further configuration, the method of the previously described implementations, wherein said one or more analog molecular structures is an analog to Structure (IX), wherein R₁ is MFA and R₂ is -NHCH₃, that comprises MFA- N-Methylnicotinamide (MFA-MeNIC) resulting in molecular structure (XIII);

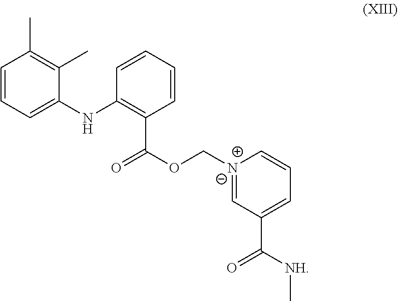

In a further configuration, the method of any of the preceding implementations, wherein one or more analog molecular structures of MFA of Structure (X) wherein R₁ is MFA and R₃ is —CH₃ that further comprises a MFA-N-methylnicotinic acid (MFA-MeNICA) molecular structure (XIV);

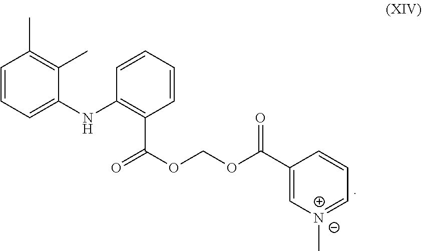

In a further configuration, the one or more analog molecules of Structure (X) wherein said one or more analog molecular structures is an analog to Structure (X), such that R₁=MFA and R₃=N-(1-methylethyl) carbamic acid, MFA-Pyridinium, 3-carboxy-1-[N-(1-methylethyl) carbamic acid] (MFA-PCA) that further comprises molecular structure (XV)

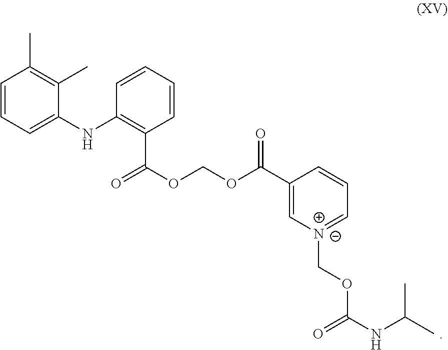

In a further configuration, the one or more mefenamic acid (MFA) analog molecules of any of the preceding implementations, wherein said molecular structures provide a basis for compositions comprising both a fenamate and analogs or derivatives thereof, including a calcium channel modulator consisting of a group of analogs or derivatives selected from one or more of gabapentin, pregabalin, and atagabalin.

In a further configuration, the one or more mefenamic acid (MFA) molecular analogs of the previously described implementations, wherein fenamate analogs or derivatives thereof are selected from one or more of a group of molecular structures consisting of MFA-NIC (XI), MFA-MeNIC (XII), MFA-2MeNIC (XIII), MFA-MeNICA (XIV) and MFA-PCA(XV).

In a further configuration, the method of the previously described implementations, wherein stability of molecular analogs of MFA immersed in water or acrylonitrile is determined by measurement on a 7^{th} day by utilizing high pressure liquid chromatography (HPLC) and wherein stability is determined by comparison of HPLC chromatographs with said molecular analogs, prior to immersion.

In a further configuration, the method of any of the preceding implementations, wherein said molecular analogs or derivatives or molecular analogs and derivatives are prodrugs.

In a further configuration, the method of any of the preceding implementations, wherein said prodrugs comprise two fenamate molecules are linked directly or linked utilizing one of a group consisting of a disulfide, palmityl, stearyl, glycol, polyethylene glycol, or ester linker.

In a further configuration, the method of the previously described implementations, further comprising administering to a subject an effective amount of a calcium channel modulator.

In a further configuration, the method of any of the preceding implementations, wherein said calcium channel modulator is gabapentin, pregabalin, or atagabalin, or an analog or derivative thereof.

In a further configuration, the method of any of the preceding implementations, wherein said prodrug comprises a calcium channel modulator molecule and a fenamate module linked directly or linked utilizing one of a group consisting of a disulfide, palmityl, stearyl, glycol, polyethylene glycol linker, or ester linker.

In a further configuration, the method of any of the preceding implementations, comprising administering to a subject an effective amount of MFA analog molecular structures consisting of a one or more of a MFA analog molecular structure group consisting of structures XI, XII, XII, XIV, and XIV. and an effective amount of gabapentin or a gabapentin prodrug, or an effective amount of a prodrug comprising said MFA analogs and gabapentin.

In a further configuration, the method of any of the preceding implementations, wherein the human subject is less than 36 months in age.

In a further configuration, the method of the previously described implementations, wherein said subject has a family history of autism or an ASD.

In a further configuration, the method of the previously described implementations, wherein said subject has one or more genetic risk factors associated with autism or an ASD.

In a further configuration or implementation of the methods and compositions described herein, in one particular implementation or embodiment, one or more pharmaceutical compositions that provide treatment for or prevention of autism or an ASD is provided, wherein said composition comprises; one or more mefenamic acid (MFA) analog molecular structures comprising structures IX or X;

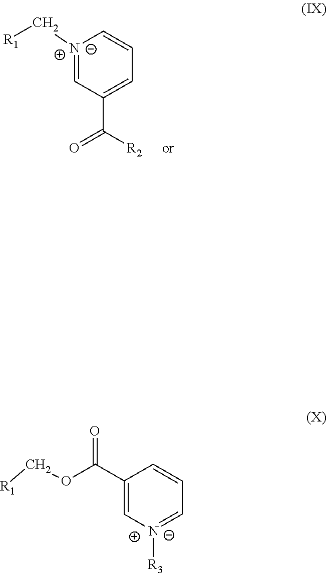

wherein R₁ is selected from the group consisting of anthranilic acid esters, including fenamic acid, MFA, tolfenamic acid (TFA), flufenamic acid (FFA), meclofenamic acid (CFA), and analogs and derivatives thereof;

R₂ is selected from the group consisting of an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, alanine, and lysine or derivatives thereof;

and R₃ is selected from the group consisting of a lower alkyl, an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, N-(1-methylethyl) carbamic acid, alanine, and lysine or derivatives thereof;

In a further configuration, the pharmaceutical compositions of the previously described implementation, wherein one or more analog molecules of MFA of molecular structure (IX) and wherein R₁ is MFA and R₂ is —NH₂, that further comprises an MFA-Nicotinamide molecule (MFA-NIC), molecular structure (XI);

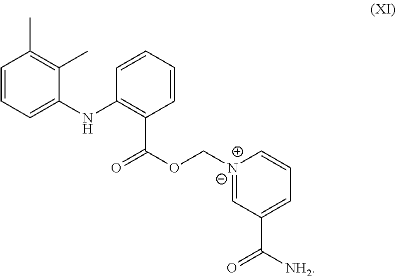

In a further configuration, the pharmaceutical compositions of any of the preceding implementations, wherein one or more analog molecules of MFA of molecular structure (IX) and wherein R₁ is MFA and R₂ is —N(CH₃)₂ that further comprises a MFA- N,N-Dimethylnicotinamide N-Methylnicotinamide (MFA-2MeNIC) molecular structure (XII);

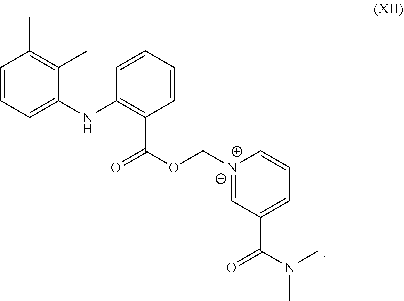

In a further configuration, the pharmaceutical compositions of any of the preceding implementations, wherein one or more analog molecules of MFA of molecular structure (IX), and wherein R₁ is MFA and R₂ is —NHCH₃, that comprises a MFA- N-Methylnicotinamide (MFA-MeNIC) molecular structure (XIII);

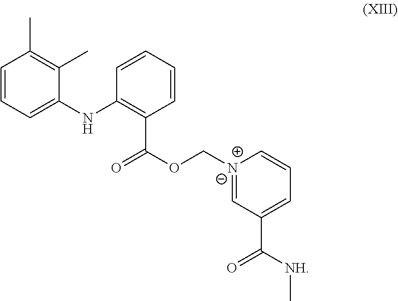

In a further configuration, the pharmaceutical compositions of any of the preceding implementations, wherein one or more analog molecules of MFA of molecular structure (X), and wherein R₁ is MFA and R₃ is —CH₃, that further comprises a MFA-N-methylnicotinic acid (MFA-MeNICA) molecular structure (XIV);

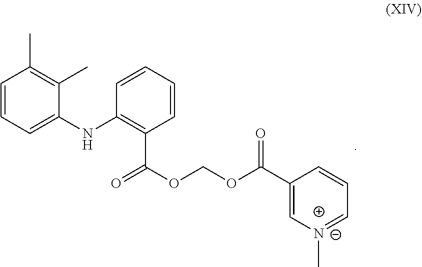

In a further configuration, the pharmaceutical compositions of any of the preceding implementationswherein one or more analog molecules of MFA of molecular structure (IX), and wherein R₁=MFA and R₃=N-(1-methylethyl) carbamic acid, MFA-Pyridinium,3-carboxy-1-[N-(1-methylethyl) carbamic acid] (MFA-PCA) further comprises molecular structure (XV).

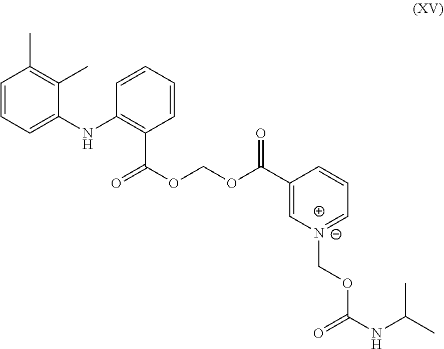

In a further configuration, the one or more mefenamic acid (MFA) analog molecular structures of any of the preceding implementations, wherein said molecular structures provide a basis for compositions comprising both a fenamate and analogs or derivatives thereof, including a calcium channel modulator consisting of a group of analogs or derivatives selected from one or more of gabapentin, pregabalin, and atagabalin.

In a further configuration, the one or more mefenamic acid (MFA) analog molecular structures of the previously described implementations, wherein said fenamate analogs or derivative thereof are selected from one or more of a group of molecular structures consisting of MFA-NIC (XI), MFA-MeNIC (XII), MFA-2MeNIC (XIII), MFA-MeNICA (XIV) and MFA-PCA(XV).

In a further configuration, the one or more mefenamic acid (MFA) analog molecular structures of any of the preceding implementations, wherein analog molecular structures MFA-NIC (XI), MFA-MeNIC (XII), and MFA-2MeNIC (XIII), exhibit solubility in distilled water and acetonitrile.

In a further configuration, the one or more mefenamic acid (MFA) analog molecular structures of any of the preceding implementations, wherein analog molecular structures MFA-NIC (XI), MFA-MeNIC (XII), and MFA-2MeNIC (XIII), exhibit stability in distilled water, saline solution, and acetonitrile.

In a further configuration, the one or more mefenamic acid (MFA) analog molecular structures of any of the preceding implementations, wherein analog molecular structures MFA-MeNICA (XIV) and MFA-MeNICA (XV) exhibit stability in acetonitrile.

In a further configuration, the one or more mefenamic acid (MFA) analog molecular structures of the foregoing implementations, wherein analog molecular structure MFA-MeNICA (XV) exhibits stability in distilled water and acetonitrile.

While this specification contains many specific embodiment details, these should not be construed as limitations on the scope of any embodiment or of what can be claimed, but rather as descriptions of features that can be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated together in a single software product or packaged into multiple software products.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this specification have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain embodiments, multitasking and parallel processing can be advantageous.

Publications and references to known registered marks representing various systems cited throughout this application are incorporated by reference herein. Citation of any above publications or documents is not intended as an admission that any of the foregoing is pertinent prior art, nor does it constitute any admission as to the contents or date of these publications or documents. All references cited herein are incorporated by reference to the same extent as if each individual publication and references were specifically and individually indicated to be incorporated by reference.

While the invention has been particularly shown and described with reference to a preferred embodiment thereof, it will be understood by those skilled in the art that various changes in form and details may be made therein without departing from the spirit and scope of the invention. As such, the invention is not defined by the discussion that appears above, but rather is defined by the claims that follow, the respective features recited in those claims, and by equivalents of such features.

1. One or more mefenamic acid (MFA) analog molecules comprising one or more molecular structures of mefenamic acid, specifically molecular structures IX or X; 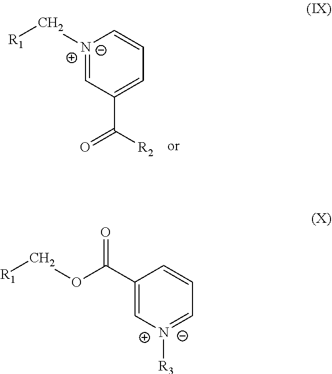
wherein R₁ is selected from the group consisting of anthranilic acid esters, including fenamic acid, MFA, tolfenamic acid (TFA), flufenamic acid (FFA), meclofenamic acid (CFA), and analogs and derivatives thereof;
R₂ is selected from the group consisting of an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, alanine, and lysine or derivatives thereof;
and R₃ is selected from the group consisting of a lower alkyl, an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, N-(1-methylethyl) carbamic acid, alanine, and lysine or derivatives thereof;

2. The one or more analog molecules of MFA of molecular structure (IX) of claim 1, wherein R₁ is MFA and R₂ is —NH₂, that comprises an MFA-Nicotinamide molecule (MFA-NIC), molecular structure (XI);

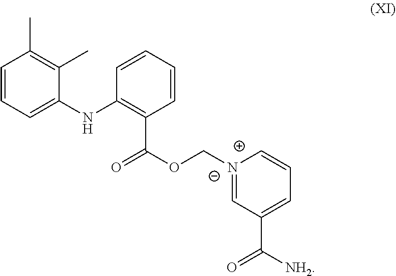

3. The one or more analog molecules of MFA of molecular Structure (IX) of claim 1, wherein R₁ is MFA and R₂ is —N(CH₃)₂ that comprises a MFA- N,N-Dimethylnicotinamide N-Methylnicotinamide (MFA-2MeNIC) molecular structure (XII);

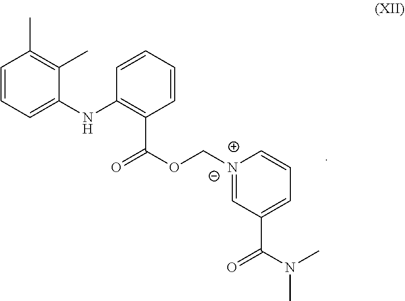 [SMILES: Cc1cccc(Nc2ccccc2C(=O)OC[n+]2cccc(C(=O)N(C)C)c2)c1C ; "."]

4. The one or more analog molecules of MFA of Structure (IX) of claim 1, wherein R₁ is MFA and R₂ is —NHCH₃, that comprises a MFA- N-Methylnicotinamide (MFA-MeNIC) molecular structure (XIII);

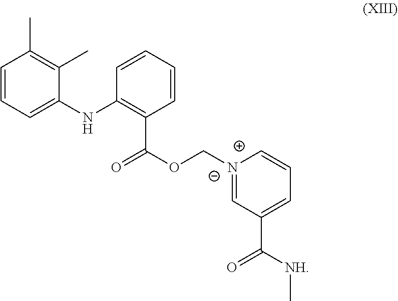

5. The one or more analog molecules of MFA of molecular Structure (X) of claim 1, wherein R₁ is MFA and R₃ is —CH₃, that comprises a MFA-N-methylnicotinic acid (MFA-MeNICA) molecular structure (XIV);

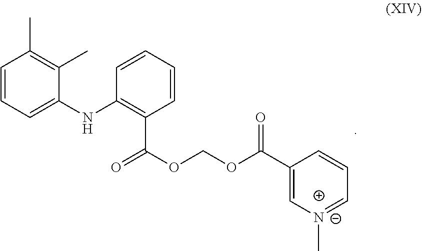

6. The one or more analog molecular Structures (X) of claim 1, wherein R₁=MFA and R₃=N-(1-methylethyl) carbamic acid, MFA-Pyridinium,3-carboxy- 1- [N-(1-methylethyl) carbamic acid] (MFA-PCA) molecular structure (XV).

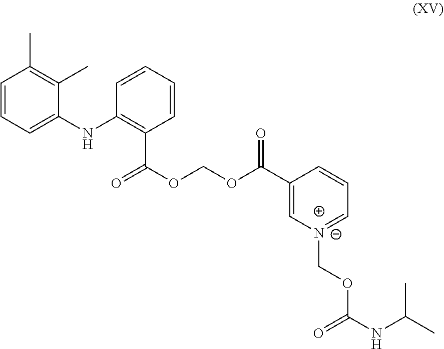

7. The one or more mefenamic acid (MFA) analog molecular structures of claim 2, wherein said molecular structures provide a basis for compositions comprising both a fenamate and analogs or derivatives thereof, including a calcium channel modulator consisting of a group of analogs or derivatives selected from one or more of gabapentin, pregabalin, and atagabalin.

8. The one or more mefenamic acid (MFA) analog molecular structures of claim 7, wherein said fenamate analogs or derivative thereof are selected from one or more of a group of molecular structures consisting of MFA-NIC (XI), MFA-MeNIC (XII), MFA-2MeNIC (XIII), MFA-MeNICA (XIV) and MFA-PCA(XV).

9. The one or more mefenamic acid (MFA) analog molecular structures of claim 8, wherein analog molecular structures MFA-NIC (XI), MFA-MeNIC (XII), and MFA-2MeNIC (XIII), exhibit solubility when immersed in distilled water and acetonitrile.

10. The one or more mefenamic acid (MFA) analog molecular structures of claim 8, wherein analog molecular structures MFA-NIC (XI), MFA-MeNIC (XII), and MFA-2MeNIC (XIII), exhibit stability when immersed in distilled water, saline solution, and acetonitrile.

11. The one or more mefenamic acid (MFA) analog molecular structures of claim 8, wherein analog molecular structures MFA-MeNICA (XIV) and MFA-MeNICA (XV) exhibit stability when immersed in acetonitrile.

12. The one or more mefenamic acid (MFA) analog molecular structures of claim 8, wherein analog molecular structure MFA-MeNICA (XV) exhibits stability when immersed in distilled water and acetonitrile.

13. A method of improving one or more indicators or symptoms of autism or an autism spectrum disorder (ASD) in a human subject, the method comprising administering to a subject exhibiting one or more indicators or symptoms of autism or an ASD, or a subject at risk of developing autism or an ASD, an effective amount of one or more analog molecular structures of MFA, wherein said analog molecular structures IX or X are; 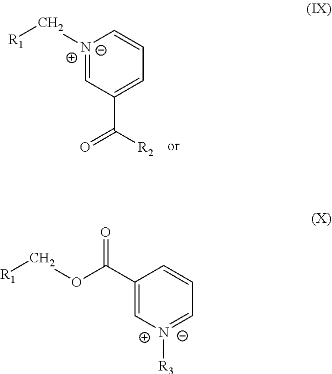
wherein R₁ is selected from the group consisting of anthranilic acid esters, including fenamic acid, MFA, tolfenamic acid (TFA), flufenamic acid (FFA), meclofenamic acid (CFA), and analogs and derivatives thereof;
R₂ is selected from the group consisting of an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, alanine, and lysine or derivatives thereof;
and R₃ is selected from the group consisting of a lower alkyl, an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, N-(1-methylethyl) carbamic acid, alanine, and lysine or derivatives thereof;

14. The method of claim 13, wherein said one or more analog molecular structures is an analog to Structure (IX), wherein R₁ is MFA and R₂ is —NH2, that further comprises an MFA-Nicotinamide molecule (MFA-NIC), resulting in molecular structure (XI);

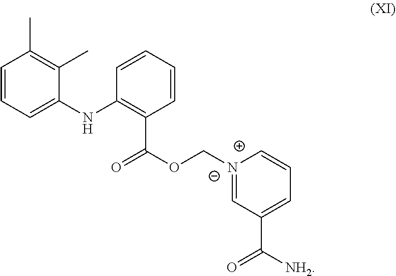

15. The method of claim 13, wherein said one or more analog molecular structures is an analog to Structure (IX), wherein R₁ is MFA and R₂ is —N(CH₃)₂ that further comprises a MFA- N,N-Dimethylnicotinamide N-Methylnicotinamide (MFA-2MeNIC) resulting in molecular structure (XII);

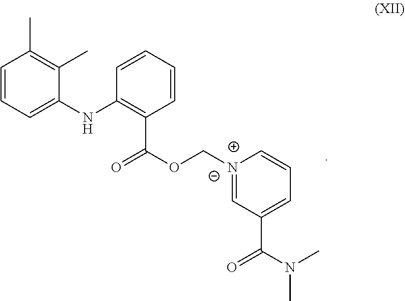

16. The method of claim 13, wherein said one or more analog molecular structures is an analog to Structure (IX), wherein R₁ is MFA and R₂ is —NHCH₃, that comprises MFA-N-Methylnicotinamide (MFA-MeNIC) resulting in molecular structure (XIII);

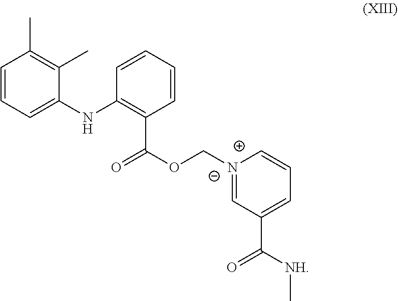

17. The method of claim 13, wherein one or more analog molecular structures of MFA of Structure (X) wherein R₁ is MFA and R₃ is —CH3 that further comprises a MFA-N-methylnicotinic acid (MFA-MeNICA) molecular structure (XIV);

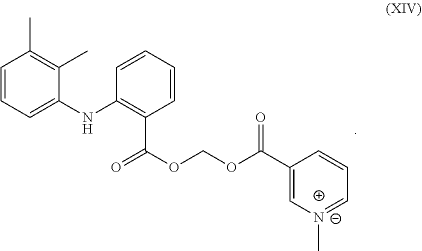

18. The one or more analog molecules of Structure (X) wherein said one or more analog molecular structures is an analog to Structure (X), such that R₁=MFA and R₃=N-(1-methylethyl) carbamic acid, MFA-Pyridinium,3-carboxy-1-[N-(1-methylethyl) carbamic acid] (MFA-PCA) that further comprises molecular structure (XV)

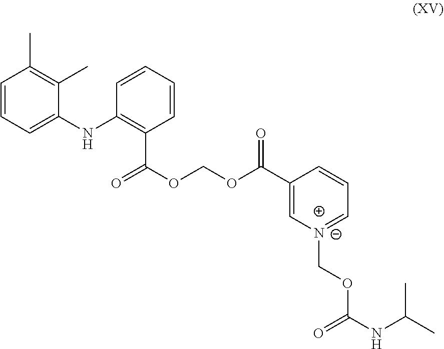

19. The one or more mefenamic acid (MFA) analog molecules of claim 14, wherein said molecular structures provide a basis for compositions comprising both a fenamate and analogs or derivatives thereof, including a calcium channel modulator consisting of a group of analogs or derivatives selected from one or more of gabapentin, pregabalin, and atagabalin.

20.-43. (canceled)

44. One or more pharmaceutical compositions that provide treatment for or prevention of autism or an ASD, wherein said composition comprises; one or more mefenamic acid (MFA) analog molecular structures comprising structures IX or X; 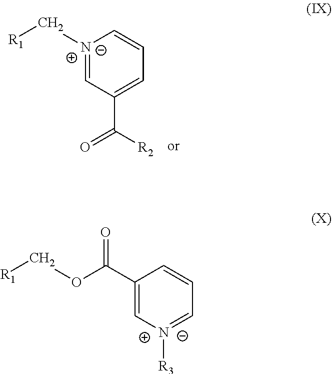
wherein R₁ is selected from the group consisting of anthranilic acid esters, including fenamic acid, MFA, tolfenamic acid (TFA), flufenamic acid (FFA), meclofenamic acid (CFA), and analogs and derivatives thereof;
R₂ is selected from the group consisting of an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, alanine, and lysine or derivatives thereof;
and R₃ is selected from the group consisting of a lower alkyl, an amide, dimethyl amine, diethyl amine, diethanol amine, dipropyl amine, pyrrolidine, piperidine, 1-methyl piperazine, N,N,N-trimethyl-1,2-ethane diamine, N,N,N-triethyl-1,2-ethane diamine, N-methyl-N,N-diethyl-1,2-ethane diamine, N-ethyl-N,N-dimethyl-1,2-ethane diamine, N-methyl-N,N-diethyl-1,3-propane diamine, N-ethyl-N,N-dimethyl-1,3-propane diamine, methyl amine, ethyl amine, 1-propyl amine, ethanol amine, 2-propyl amine, 1-butyl amine, 2-butyl amine, 2-methyl-2-propyl amine, piperazine, N,N-dimethyl-ethyl diamine, N,N-diethyl-ethyl diamine, N,N-dimethyl propyl diamine, N,N-diethyl-propyl diamine, N,N-dimethyl amino propylene amine, N,N-dimethyl ethylene amine, N,N-diethyl amino propylene amine, N,N-diethylamino ethylene amine, amino ethyl-piperazine, N-methyl-1,2-ethane diamine, N-ethyl-1,2-ethane diamine, N-methyl-1,3-propane diamine, N-ethyl-1,3-propane diamine, 1,2-diamine ethane, 1,3-diamino propane, 1,4-diamino butane, cadaverine, cystamine, 1,6-diamino hexane, 1,2-diamine benzene, 1,3-diamino benzene, 1,4-diamino benzene, 1,4-diamino butanol, 4,4-diamino-3-hydroxy butanoic acid, 5-amino-1,3,3-trimethylcyclohexanemethylamine,2,2′-oxybis ethanamine, N-(1-methylethyl) carbamic acid, alanine, and lysine or derivatives thereof;
